(19) United States
(12) Patent Application Publication
(10) Pub. No.: US 2019/0001272 A1
(43) Pub. Date: Jan. 3, 2019

(54) Silk Solution Purification System, Concentrating System, and Methods Thereof

(71) Applicant: TUFTS UNIVERSITY, Medford, MA (US)

(72) Inventors: Gary G. Leisk, Somerville, MA (US); David L. Kaplan, Concord, MA (US); Benjamin P. Partlow, Winchester, MA (US); Tim Jia-Ching Lo, Taoyuan City (TW)

(21) Appl. No.: 16/063,485

(22) PCT Filed: Dec. 16, 2016
(86) PCT No.: PCT/US2016/067151

§ 371 Date: Jun. 18, 2018

Related U.S. Application Data
(60) Provisional application No. 62/269,779, filed on Dec. 18, 2015.

(51) Int. Cl. B01D 61/24; B01D 61/28; B01D 61/58; B01D 63/06; B01D 61/32; B01D 71/10; C07K 14/435

(52) U.S. Cl. CPC B01D 61/243; B01D 61/28; B01D 61/58; B01D 63/06; D01B 7/00; B01D 71/10; C07K 14/43518; B01D 2317/022; D10B 2211/22; B01D 61/32

(57) ABSTRACT

The present disclosure provides, among other things, systems for processing silk. Provided systems purify silk fibroin solutions without inducing conformational changes in the silk proteins. Provided systems concentrate silk fibroin solutions. The present disclosure also provides methods of purifying and concentrating silk fibroin solutions. Provided systems and methods are useful for processing silk fibroin for any application.

### CROSS-REFERENCE TO RELATED APPLICATION

This patent application claims the benefit of priority of U.S. patent application No. 62/269,779, filed on Dec. 18, 2015, the contents of which is hereby incorporated by reference in its entirety for all purposes herein.

### GOVERNMENT SUPPORT

This invention was made with government support under grant number EB002520 awarded by the National Institutes of Health. The government has certain rights in the invention.

### BACKGROUND

Silk is a natural fiber produced by silkworms and spiders. Silk fibroin and specifically solutions of silk fibroin can be processed to form various materials and structures with unique mechanical and optical properties. In conjunction with silk's biocompatibility and degradability, silk is an attractive option for use in a wide array of applications, such as, for example, biomaterials, biomedical devices, commercial products, electronics, pharmaceutical products, photonics, robotics, sensing, or tissue engineering applications.

### SUMMARY

Among other things, the present disclosure provides apparatus and systems useful for processing silk. The present disclosure provides methods of using such apparatus and systems for processing silk, including for example to generate and/or utilize purified and/or concentrated silk fibroin solutions.

In some embodiments, the present disclosure includes systems and methods for purifying and/or concentrating silk solutions, for example for use silk fibroin applications.

As noted herein, silk fibroin, and solutions thereof, is known to be useful in a wide variety of applications including, for example, for use as and/or incorporation into: anti-counterfeiting materials, biomaterials, biomedical devices, commercial products, controlled degradation applications, controlled release applications, drug delivery devices or materials, drug release devices or materials, electronics, extrusion injection molding, materials for tunable degradation, optics, photonics, materials that protect, preserve and/or stabilize biologically labile and/or heat labile agents, prosthetics, tissue scaffolds [e.g., as may be used in tissue engineering and/or regeneration applications], robotic devices or materials, sensors, and/or wound healing bandages or hydrogels etc.

A variety of technologies for processing silk fibroin solutions for use in such applications are also known in the art, including for example by gelling (including by electro gelling, sonicating, and/or vortexing), by molding (including injection molding such as extrusion injection molding), by printing, or spinning (including electrospinning).

In some embodiments, silk processing in accordance with the present disclosure includes multiple steps. In some embodiments, silk processing includes providing a silk source, for example Bombyx mori. In some embodiments, silk processing includes degumming a raw silk source to remove sericin, a glue-like outer protein that covers silk fibroin. In some embodiments, silk processing includes rinsing and/or drying silk fibroin. In some embodiments, silk processing includes dissolving silk fibroin to form a silk fibroin solution. In some embodiments, a dissolving step includes adding silk fibroin to a salt solution, such as for example, a lithium bromide solution. In some embodiments, a silk fibroin solution includes or consists of dissolved silk fibroin, and may optionally include salts, solvent, contaminants and/or ions.

In some embodiments, provided systems purify silk fibroin solutions to remove impurities such as salts, solvent, contaminants and/or ions. Alternatively or additionally, in some embodiments, provided systems concentrate silk fibroin solutions. In certain embodiments, purification and/or concentration as achieved by embodiments of the present disclosure generate silk solutions particularly useful for certain applications and/or enable new applications for silk solutions by generating uniquely pure and/or concentrated solutions.

The present disclosure encompasses a recognition that silk fibroin solutions can be highly sensitive to protein aggregation. The present disclosure also encompasses a recognition that silk fibroin proteins present in silk fibroin solutions tend to aggregate when exposed to shear stress. Shear stress can be caused, for example, by torsion on a closed, thin-walled tube, such as when a silk fibroin solution passes or flows through such a thin passage. The present disclosure also encompasses a recognition that traditional apparatus and methods used for protein purification and/or concentration exhibit high levels of shear that induce protein aggregation in dissolved silk fibroin solutions. The present disclosure therefore identifies a source of a problem (risk of aggregation) with certain traditional apparatus and methods often used for protein purification and/or concentration.

In some embodiments, the present disclosure provides systems for purifying and/or concentrating silk fibroin solutions. In some embodiments, the present disclosure provides systems for automated preparation of purified and/or concentrated silk fibroin solutions.

In some embodiments, the present disclosure provides systems for purifying silk fibroin solutions that include or consist of dissolved silk fibroin. In some embodiments, the present disclosure provides systems for automated preparation of purified and/or concentrated solutions that include or consist of dissolved silk fibroin, and optionally include one or more salts, solvent, contaminants and/or ions. In some embodiments, an automated silk fibroin purification system as described herein purifies a silk fibroin solution by removing salts, solvent, contaminants and/or ions therefrom.

In some embodiments, a silk fibroin solution generated and/or utilized in accordance with the present disclosure contains >1% w/v of silk fibroin. In some embodiments, a silk fibroin solution generated and/or utilized in accordance with the present disclosure has a viscosity between about 1 cP and about 50 cP. In some embodiments, a silk fibroin solution generated and/or utilized in accordance with the present disclosure contains salts, solvent, contaminants and/or ions.

In some embodiments, provided technologies permit purification and/or concentration of a silk solution without requiring its prior dilution. In some embodiments, provided technologies permit purification and/or concentration of a silk solution without requiring prior steps of removing salts, solvents, contaminants and/or ions. For example in some embodiments, a silk solution including one or more salts, solvents, contaminants and/or ions is directly input in a provided silk solution purification system.

In some embodiments, provided silk solution purification and/or concentration systems dialyze a dissolved silk fibroin solution without either clogging a porous membrane or generating shear-induced conformation changes in silk fibroin proteins.

In some embodiments, automated systems as described herein for preparation of purified and/or concentrated silk fibroin solutions operate with minimal oversight. In some embodiments, automated systems operate without requiring operator intervention, for example in changing solvent (e.g., water) and/or in changing a porous membrane.

In some embodiments, provided silk solution purification and/or concentration systems purify higher concentration silk fibroin solutions without clogging or generating shear-induced conformation changes and removing salts, solvent, contaminants and/or ions both more effectively and more efficiently than existing protein purification systems.

In some embodiments, provided systems reduce bromine concentrations by a factor of 10-fold more than existing systems and reduce lithium concentrations by a factor of 1.8-fold more than existing systems.

In some embodiments, provided systems achieve purification and/or concentration as described herein within 24 hours and/or with yields in excess of 500 mL.

In some embodiments, provided silk processing systems (including, e.g., automated systems) include a dual-chamber element.

In some embodiments, a silk fibroin solution is introduced, enters, or flows into a dual-chamber element of a silk fibroin purification system as described herein (e.g., an automated system).

In some embodiments, a dual-chamber element includes first and second chambers.

In some embodiments, an arrangement, configuration, size, and shape of a dual-chamber element is defined by first and second chambers. In some embodiments, first and second chambers can be of any of a variety of sizes and/or shapes. In some embodiments, one or both of the first and second chambers are substantially tubular, such that each is about circular and elongated. In some embodiments, first and second chambers are the same length. In some embodiments, first and second chambers are different lengths.

In some embodiments, a first chamber and a second chamber are adjacent to one another or abut one another. In some embodiments, first and second chambers are separated by a common surface or wall. In some embodiments, first and second chambers share at least one surface or wall in common. In some embodiments, at least one common surface or wall between a first chamber and a second chamber is porous. In some embodiments, a common or shared wall or surface is defined by a porous membrane. In some embodiments, first and second chambers are separated by a porous membrane.

In some embodiments, a second chamber substantially surrounds a first chamber so that the second and first chambers are outer and inner chambers, respectively. In some embodiments, a first chamber is enclosed within or by a second chamber. In some embodiments, an outer surface of a first chamber is a common surface or wall between a first chamber and a second chamber.

In some embodiments, a common or shared wall or surfaces is defined by or separated by a porous membrane. In some embodiments, a tubular porous membrane surrounds and defines a first chamber. In some embodiments, a first chamber's shape is defined by a porous membrane. In some embodiments, a rigid outer tube surrounds a tubular porous membrane to create a second chamber.

In some embodiments, a first chamber includes a rigid porous tube. In some embodiments, a dissolved silk solution is introduced through a rigid porous tube. In some embodiments, a dissolved silk solution enters a first chamber at an entrance to a rigid porous tube. In some embodiments, a dissolved silk solution flows through holes in a rigid tube and fills a first chamber. In some embodiments, a dissolved silk solution exits a first chamber at an exit to a rigid porous tube. In some embodiments, a rigid porous tube provides support for a first chamber.

In some embodiments, a porous membrane includes pores size to retain proteins above about 1 kDa. In some embodiments, a porous membrane is or includes one or more members selected from a group consisting of a semi-permeable membrane, a selectively permeable membrane, a dialysis membrane, cellulose tubing, regenerated cellulose tubing, or SnakeSkin tubing.

In some embodiments, when dissolved silk fibroin solution is introduced, enters, or flows into a dual-chamber element it flows at a rate between about 0.01 ml per minute to about 0.5 ml per minute.

In some embodiments, a dissolved silk fibroin solution is introduced, enters, or flows through a first chamber with a positive pressure. In some embodiments, a dissolved silk fibroin solution is introduced, enters, or flows through a first chamber with a positive pressure relative to a pressure of a second chamber.

In some embodiments, a transmembrane pressure is an average pressure differential between a first chamber and a second chamber. In some embodiments, a transmembrane pressure is a force that pushes salts, contaminants, solvents, and/or ions from a first chamber through a porous membrane to a second chamber. In some embodiments, a transmembrane pressure is between about 0.10 psi-about 50 psi.

In some embodiments, viscosity, flow, pressure, temperature vary with a geometry of a dual-chamber element. In some embodiments, geometric dimensions includes length, width, and depth of first and second chambers.

In some embodiments, geometry includes a ratio of surface area to retained volume. In some embodiments, the present disclosure provides systems including a smaller ratio of surface area to retained volume. In some embodiments, a smaller ratio results in a gap between a porous membrane and an outer wall.

In some embodiments, geometry includes a gap between a porous membrane and an outside wall of a second chamber. In some embodiments, a gap is defined as a distance between a porous membrane separating an inner wall of a second chamber and an outer wall of a porous membrane. In some embodiments, a gap is between about less that a millimeter and several millimeters. In some embodiments, a gap is up to about 20 mm. In some embodiments, a gap reduces flow and pressure. In some embodiments, a gap reduces shear sensitivity in a dissolved silk fibroin solution. In some embodiments, a gap reduces a tendency of a dissolved silk fibroin solution to form large aggregates.

In some embodiments, a dual-chamber element is dimensioned and arranged so that when a dissolved silk fibroin solution travels into or through a first chamber, salts, contaminants, solvents, and/or ions from a dissolved silk fibroin solution cross a porous membrane and into a second chamber.

In some embodiments, a dual-chamber element is dimensioned and arranged so that when a dissolved silk fibroin solution travels into or through a first chamber, salts, contaminants, solvents, and/or ions from a dissolved silk fibroin solution cross a porous membrane and into a dialysate in a second chamber. In some embodiments, a dialysate is fluid. In some embodiments, a dialysate is water. In some embodiments, a fluid in a second chamber is a counter-flow fluid. In some embodiments, a counter flow fluid flows in a second chamber in a direction that opposes a flow of a dissolved silk fibroin solution in a first chamber.

In some embodiments, silk proteins from a dissolved silk fibroin solution are retained in a retentate in a first chamber. In some embodiments a purified silk fibroin solution is retained in a first chamber. In some embodiments a purified silk fibroin solution flows out of a first chamber.

In some embodiments, air pockets present in a first chamber cause a buildup of pressure. In some embodiments, increased pressure may induce shear. In some embodiments, a vacuum pump removes air pockets. In some embodiments, removing air pockets reduces pressure buildup thereby reducing the likelihood of shear.

In some embodiments, salts, contaminants, solvents, and/or ions may collect near the bottom of a second chamber reducing exposed surface area of a porous membrane. In some embodiments, such collecting reduces efficiency. In some embodiments, a dual-chamber element is tilted from normal and reduces salts, contaminants, solvents, and/or ions collecting. In some embodiments, a dual-chamber element is tilted for example at about 45° from normal.

In some embodiments, provided automated silk purification systems include at least one dual-chamber element. In some embodiments, provided automated silk purification systems include multiple dual-chamber elements.

In some embodiments, provided automated silk purification systems include two or more dual-chamber elements, the elements are connected in series. In some embodiments, provided automated silk purification systems include two or more dual-chamber elements, the elements are connected but operate in parallel to one another.

In some embodiments, provided automated silk purification systems include a mixing stage or reservoir arranged at an output of a first dual-chamber element.

In some embodiments, provided automated silk purification systems include two or more dual-chamber elements, each dual-chamber element is the same length or about the same length. In some embodiments, provided automated silk purification systems include two or more dual-chamber elements, each dual-chamber elements is a different length, for example a first dual-chamber element is 30 cm and a second dual-chamber element is 100 cm.

In some embodiments, silk solution purification systems include cameras, chemical analysis equipment, sensors, and/or techniques to measure silk solution properties, for example, silk concentration, salt concentration, ion concentration, a concentration of higher order configurations of silk, and/or turbidity. In some embodiments, silk solution purification systems include cameras, chemical analysis equipment, sensors, and/or techniques to measure silk solution properties in situ.

In some embodiments, a purified silk fibroin solution is stored in a reservoir. In some embodiments, a stored purified silk fibroin solution is fed to a concentrating system. In some embodiments, an automated silk purification system is integrated with a silk fibroin solution concentrating system.

In some embodiments, provided automated silk concentrating systems include a dual-chamber element. In some embodiments, an automated silk concentrating system is in parallel with an automated silk purification system. In some embodiments, an automated silk concentrating system is in series with an automated silk purification system. In some embodiments, when an automated silk concentrating system operates in series with an automated silk purification system, an automated silk concentrating system is a terminal system.

In some embodiments, a purified silk fibroin solution has a concentration for example between about 1% w/v and about 5% w/v. In some embodiments, provided silk concentration systems produce a concentrated purified silk fibroin solution between about 1% w/v and about 50% w/v.

In some embodiments, provided silk solution purification and/or concentration systems including a dual chamber and/or porous membrane to selectively produce or retain silk proteins with a particular molecular weight or having a narrow range of molecular weights to produce a monodisperse silk solution. In some embodiments, a narrow range of molecular weights is a population centered about an average molecular weight. In some embodiments, a narrow range of molecular weights is a population distributed within the narrow range. In some embodiments, a population distributed within the range is uniformly distributed or non-uniformly distributed. In some embodiments, provided silk solution purification and/or concentration systems form a silk solution having a polydispersity index of less than about 0.4.

In some embodiments, a dual-chamber element is vertical. In some embodiments, when a purified silk fibroin solution is fed into a first chamber from a top of a dual-chamber element. In some embodiments, when a purified silk fibroin solution is gravity fed into a first chamber from a top of a dual-chamber element. In some embodiments, a vertical arrangement and gravity-fed design of a silk solution concentrating system reduces shear relative to prior designs.

In some embodiments, a second chamber includes or is filled with air or a gas. In some embodiments, a second chamber includes no water or other fluids.

In some embodiments, a solvent, such as for example water present in a purified silk fibroin solution that is contained in a first chamber crosses a porous membrane into a second chamber. In some embodiments, a purified silk fibroin solution is retained in a first chamber. In some embodiments, a concentrated purified silk fibroin solution is retained in a first chamber.

In some embodiments, a vertical arrangement and gravity-fed design of a silk solution concentrating system automatically separates a concentrated purified silk fibroin solution.

In some embodiments, a silk solution concentrating system includes a valve at an opening for introducing a purified silk fibroin solution. In some embodiments, a silk solution concentrating system includes at least one valve at or near a bottom of a dual-chamber element for removing a concentrated purified silk fibroin solution. In some embodiments, a silk solution concentrating system includes multiple valves along a side of a dual-chamber element for removing different concentrations of a concentrated purified silk fibroin solution. In some embodiments, different concentrations of silk are extracted based on the height where a sample is present in a column by extracting through a valve at such a location.

In some embodiments, a silk solution concentrating systems include sensors or chemical analysis equipment and techniques to measure silk solution properties, for example, silk concentration, salt concentration, ion concentration, a concentration of higher order configurations of silk, and/or turbidity.

In some embodiments, the present disclosure provides methods for automated preparation of purified silk fibroin solutions. In some embodiments, the present disclosure provides methods for automated preparation of concentrated purified silk fibroin solutions.

In some embodiments, methods include providing a dissolved silk fibroin solution for purification. In some embodiments, a dissolved silk fibroin solution includes salts, solvents, contaminants, and/or ions.

In some embodiments, methods include introducing or flowing a dissolved silk fibroin solution into a first chamber of an automated silk purification system. In some embodiments, methods include introducing or flowing a dissolved silk fibroin solution through a first chamber of an automated silk purification system.

In some embodiments, methods include contacting a dissolved silk fibroin solution with a porous membrane. In some embodiments, methods include flowing a dissolved silk fibroin solution over a porous membrane. In some embodiments, methods include pumping a dissolved silk fibroin solution into a first chamber of an automated silk purification system.

In some embodiments, a dissolved silk fibroin solution introducing or flowing through a first chamber is characterized by a pressure and a flow rate. In some embodiments, a pressure and/or flow rate of a dissolved silk fibroin solution is below a threshold that induces silk protein aggregation.

In some embodiments, methods include providing a fluid in a second chamber of an automated silk purification system. In some embodiments, a fluid is water. In some embodiments, methods include introducing or flowing a fluid through a second chamber of an automated silk purification system. In some embodiments, a counter-flow fluid flows in a second chamber in a direction that opposes a flow of a dissolved silk fibroin solution in a first chamber.

In some embodiments, methods include retaining silk proteins in a dissolved silk fibroin solution that is in a first chamber or that is flowing through a first chamber. In some embodiments, methods include extracting a fluid from a second chamber including salts, solvent, contaminants, and/or ions that entered or crossed a porous membrane separating first and second chambers.

In some embodiments, methods include providing an automated silk purification system including at least two dual-chamber elements. In some embodiments, methods include providing an automated silk purification system including at least two dual-chamber elements where each dual-chamber element is a same length. In some embodiments, methods include providing an automated silk purification system including at least two dual-chamber elements where at least one dual-chamber element is a different length. In some embodiments, methods include connecting dual-chamber elements in parallel or in series.

In some embodiments, methods include tilting each dual-chamber element away from normal.

In some embodiments, methods include detecting, in situ detecting and/or monitoring a silk solution concentration, a salt concentration, an ion concentration, a concentration of higher order configurations of silk, and/or a silk solution turbidity. In some embodiments, methods include in situ detecting a silk solution concentration, a salt concentration, an ion concentration, a concentration of higher order configurations of silk, and/or a silk solution turbidity.

In some embodiments, methods include introducing a silk fibroin solution (e.g., a purified solution) into a first chamber of a dual-chamber element of a silk solution concentrating system. In some embodiments, methods include gravity feeding a silk fibroin solution into a first chamber of a dual-chamber element of a silk solution concentrating system. In some embodiments, methods include providing a fluid in a second chamber of a silk solution concentrating system. In some embodiments, a fluid is a gas. In some embodiments, a gas is air.

In some embodiments, methods include extracting a fluid from a second chamber including a solvent that entered or crossed a porous membrane separating first and second chambers. In some embodiments, methods include retaining silk proteins in a purified silk fibroin solution in a first chamber.

In some embodiments, methods include detecting, in situ detecting and/or monitoring a silk solution concentration. In some embodiments, methods include detecting, in situ detecting and/or monitoring using cameras, chemical analysis equipment, and/or sensors.

### BRIEF DESCRIPTION OF THE DRAWING

The foregoing and other objects, aspects, features, and advantages of the present disclosure will become more apparent and better understood by referring to the following description taken in conjunction with the accompanying figures in which:

FIG. 1 is a flow chart that shows silk processing stages.

FIG. 2 shows a silk purification system of some embodiments.

FIG. 3 shows a silk purification system of some embodiments.

FIG. 4 shows a silk purification system in accordance with some embodiments.

FIG. 5 shows a silk concentration system of some embodiments.

### DEFINITIONS

In order for the present disclosure to be more readily understood, certain terms are first defined below. Additional definitions for the following terms and other terms are set forth throughout the specification.

In this application, unless otherwise clear from context, the term “a” may be understood to mean “at least one.” As used in this application, the term “or” may be understood to mean “and/or.” In this application, the terms “including” and “including” may be understood to encompass itemized components or steps whether presented by themselves or together with one or more additional components or steps. Unless otherwise stated, the terms “about” and “approximately” may be understood to permit standard variation as would be understood by those of ordinary skill in the art. Where ranges are provided herein, the endpoints are included. As used in this application, the term “include” and variations of the term, such as “including” and “includes,” are not intended to exclude other additives, components, integers or steps.

As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Affinity”: As is known in the art, “affinity” is a measure of the tightness with a particular ligand binds to its partner. Affinities can be measured in different ways. In some embodiments, affinity is measured by a quantitative assay. In some such embodiments, binding partner concentration may be fixed to be in excess of ligand concentration so as to mimic physiological conditions. Alternatively or additionally, in some embodiments, binding partner concentration and/or ligand concentration may be varied. In some such embodiments, affinity may be compared to a reference under comparable conditions (e.g., concentrations).

“Agent”: As used herein, the term “agent” may refer to a compound or entity of any chemical class including, for example, polypeptides, nucleic acids, saccharides, lipids, small molecules, metals, or combinations thereof. As will be clear from context, in some embodiments, an agent can be or include a cell or organism, or a fraction, extract, or component thereof. In some embodiments, an agent is agent is or includes a natural product in that it is found in and/or is obtained from nature. In some embodiments, an agent is or includes one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents are provided as collections or libraries, for example that may be screened to identify or characterize active agents within them. Some particular embodiments of agents that may be utilized in accordance with the present disclosure include small molecules, antibodies, antibody fragments, aptamers, siRNAs, shRNAs, DNA/RNA hybrids, antisense oligonucleotides, ribozymes, peptides, peptide mimetics, small molecules, etc. In some embodiments, an agent is or includes a polymer. In some embodiments, an agent is not a polymer and/or is substantially free of any polymer. In some embodiments, an agent contains at least one polymeric moiety. In some embodiments, an agent lacks or is substantially free of any polymeric moiety.

“Analog”: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance

“Amino acid”: As used herein, the term “amino acid,” in its broadest sense, refers to any compound and/or substance that can be incorporated into a polypeptide chain, e.g., through formation of one or more peptide bonds. In some embodiments, an amino acid has the general structure H2N—C(H)(R)—COOH. In some embodiments, an amino acid is a naturally-occurring amino acid. In some embodiments, an amino acid is a synthetic amino acid; in some embodiments, an amino acid is a D-amino acid; in some embodiments, an amino acid is an L-amino acid. “Standard amino acid” refers to any of the twenty standard L-amino acids commonly found in naturally occurring peptides. “Nonstandard amino acid” refers to any amino acid, other than the standard amino acids, regardless of whether it is prepared synthetically or obtained from a natural source. In some embodiments, an amino acid, including a carboxy-and/or amino-terminal amino acid in a polypeptide, can contain a structural modification as compared with the general structure herein. For example, in some embodiments, an amino acid may be modified by methylation, amidation, acetylation, and/or substitution as compared with the general structure. In some embodiments, such modification may, for example, alter the circulating half-life of a polypeptide containing the modified amino acid as compared with one containing an otherwise identical unmodified amino acid. In some embodiments, such modification does not significantly alter a relevant activity of a polypeptide containing the modified amino acid, as compared with one containing an otherwise identical unmodified amino acid. As will be clear from context, in some embodiments, the term “amino acid” is used to refer to a free amino acid; in some embodiments it is used to refer to an amino acid residue of a polypeptide.

“Associated” or “Associated with”: As used herein, the term “associated” or “associated with” typically refers to two or more entities in physical proximity with one another, either directly or indirectly (e.g., via one or more additional entities that serve as a linking agent), to form a structure that is sufficiently stable so that the entities remain in physical proximity under relevant conditions, e.g., physiological conditions. In some embodiments, associated entities are covalently linked to one another. In some embodiments, associated entities are non-covalently linked. In some embodiments, associated entities are linked to one another by specific non-covalent interactions (i.e., by interactions between interacting ligands that discriminate between their interaction partner and other entities present in the context of use, such as, for example, streptavidin/avidin interactions, antibody/antigen interactions, etc.). Alternatively or additionally, a sufficient number of weaker non-covalent interactions can provide sufficient stability for moieties to remain associated. Exemplary non-covalent interactions include, but are not limited to, affinity interactions, metal coordination, physical adsorption, host-guest interactions, hydrophobic interactions, pi stacking interactions, hydrogen bonding interactions, van der Waals interactions, magnetic interactions, electrostatic interactions, dipole-dipole interactions, etc.

“Binding”: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts—including where interacting entities or moieties are studied in isolation or in the context of more complex systems (e.g., while covalently or otherwise associated with a carrier entity and/or in a biological system or cell).

“Binding agent”: In general, the term “binding agent” is used herein to refer to any entity that binds to a target of interest as described herein. In many embodiments, a binding agent of interest is one that binds specifically with its target in that it discriminates its target from other potential binding partners in a particular interaction contect. In general, a binding agent may be or include an entity of any chemical class (e.g., polymer, non-polymer, small molecule, polypeptide, carbohydrate, lipid, nucleic acid, etc). In some embodiments, a binding agent is a single chemical entity. In some embodiments, a binding agent is a complex of two or more discrete chemical entities associated with one another under relevant conditions by non-covalent interactions. For example, those skilled in the art will appreciate that in some embodiments, a binding agent may include a “generic” binding moiety (e.g., one of biotin/avidin/streptaviding and/or a class-specific antibody) and a “specific” binding moiety (e.g., an antibody or aptamers with a particular molecular target) that is linked to the partner of the generic biding moiety. In some embodiments, such an approach can permit modular assembly of multiple binding agents through linkage of different specific binding moieties with the same generic binding poiety partner. In some embodiments, binding agents are or include polypeptides (including, e.g., antibodies or antibody fragments). In some embodiments, binding agents are or include small molecules. In some embodiments, binding agents are or include nucleic acids. In some embodiments, binding agents are aptamers. In some embodiments, binding agents are polymers; in some embodiments, binding agents are not polymers. In some embodiments, binding agents are non-polymeric in that they lack polymeric moieties. In some embodiments, binding agents are or include carbohydrates. In some embodiments, binding agents are or include lectins. In some embodiments, binding agents are or include peptidomimetics. In some embodiments, binding agents are or include scaffold proteins. In some embodiments, binding agents are or include mimeotopes. In some embodiments, binding agents are or include stapled peptides. In certain embodiments, binding agents are or include nucleic acids, such as DNA or RNA.

“Biocompatible”: The term “biocompatible”, as used herein, refers to materials that do not cause significant harm to living tissue when placed in contact with such tissue, e.g., in vivo. In certain embodiments, materials are “biocompatible” if they are not toxic to cells. In certain embodiments, materials are “biocompatible” if their addition to cells in vitro results in less than or equal to 20% cell death, and/or their administration in vivo does not induce significant inflammation or other such adverse effects.

“Biodegradable”: As used herein, the term “biodegradable” refers to materials that, when introduced into cells, are broken down (e.g., by cellular machinery, such as by enzymatic degradation, by hydrolysis, and/or by combinations thereof) into components that cells can either reuse or dispose of without significant toxic effects on the cells. In certain embodiments, components generated by breakdown of a biodegradable material are biocompatible and therefore do not induce significant inflammation and/or other adverse effects in vivo. In some embodiments, biodegradable polymer materials break down into their component monomers. In some embodiments, breakdown of biodegradable materials (including, for example, biodegradable polymer materials) involves hydrolysis of ester bonds. Alternatively or additionally, in some embodiments, breakdown of biodegradable materials (including, for example, biodegradable polymer materials) involves cleavage of urethane linkages. Exemplary biodegradable polymers include, for example, polymers of hydroxy acids such as lactic acid and glycolic acid, including but not limited to poly(hydroxyl acids), poly(lactic acid)(PLA), poly(glycolic acid)(PGA), poly(lactic-co-glycolic acid)(PLGA), and copolymers with PEG, polyanhydrides, poly(ortho)esters, polyesters, polyurethanes, poly(butyric acid), poly(valeric acid), poly(caprolactone), poly(hydroxyalkanoates, poly(lactide-co-caprolactone), blends and copolymers thereof. Many naturally occurring polymers are also biodegradable, including, for example, proteins such as albumin, collagen, gelatin and prolamines, for example, zein, and polysaccharides such as alginate, cellulose derivatives and polyhydroxyalkanoates, for example, polyhydroxybutyrate blends and copolymers thereof. Those of ordinary skill in the art will appreciate or be able to determine when such polymers are biocompatible and/or biodegradable derivatives thereof (e.g., related to a parent polymer by substantially identical structure that differs only in substitution or addition of particular chemical groups as is known in the art).

“Biologically active”: As used herein, the phrase “biologically active” refers to a substance that has activity in a biological system (e.g., in a cell (e.g., isolated, in culture, in a tissue, in an organism), in a cell culture, in a tissue, in an organism, etc.). For instance, a substance that, when administered to an organism, has a biological effect on that organism, is considered to be biologically active. It will be appreciated by those skilled in the art that often only a portion or fragment of a biologically active substance is required (e.g., is necessary and sufficient) for the activity to be present; in such circumstances, that portion or fragment is considered to be a “biologically active” portion or fragment.

“Characteristic portion”: As used herein, the term “characteristic portion” is used, in the broadest sense, to refer to a portion of a substance whose presence (or absence) correlates with presence (or absence) of a particular feature, attribute, or activity of the substance. In some embodiments, a characteristic portion of a substance is a portion that is found in the substance and in related substances that share the particular feature, attribute or activity, but not in those that do not share the particular feature, attribute or activity. In certain embodiments, a characteristic portion shares at least one functional characteristic with the intact substance. For example, in some embodiments, a “characteristic portion” of a protein or polypeptide is one that contains a continuous stretch of amino acids, or a collection of continuous stretches of amino acids, that together are characteristic of a protein or polypeptide. In some embodiments, each such continuous stretch generally contains at least 2, 5, 10, 15, 20, 50, or more amino acids. In general, a characteristic portion of a substance (e.g., of a protein, antibody, etc.) is one that, in addition to the sequence and/or structural identity specified above, shares at least one functional characteristic with the relevant intact substance. In some embodiments, a characteristic portion may be biologically active.

“Comparable”: The term “comparable”, as used herein, refers to two or more agents, entities, situations, sets of conditions, etc. that may not be identical to one another but that are sufficiently similar to permit comparison therebetween so that conclusions may reasonably be drawn based on differences or similarities observed. Those of ordinary skill in the art will understand, in context, what degree of identity is required in any given circumstance for two or more such agents, entities, situations, sets of conditions, etc. to be considered comparable.

“Conjugated”: As used herein, the terms “conjugated,” “linked,” “attached,” and “associated with,” when used with respect to two or more moieties, means that the moieties are physically associated or connected with one another, either directly or via one or more additional moieties that serves as a linking agent, to form a structure that is sufficiently stable so that the moieties remain physically associated under the conditions in which structure is used, e.g., physiological conditions. Typically the moieties are attached either by one or more covalent bonds or by a mechanism that involves specific binding. Alternately, a sufficient number of weaker interactions can provide sufficient stability for moieties to remain physically associated.

“Corresponding to”: As used herein, the term “corresponding to” is often used to designate the position/identity of a residue in a polymer, such as an amino acid residue in a polypeptide or a nucleotide residue in a nucleic acid. Those of ordinary skill will appreciate that, for purposes of simplicity, residues in such a polymer are often designated using a canonical numbering system based on a reference related polymer, so that a residue in a first polymer “corresponding to” a residue at position 190 in the reference polymer, for example, need not actually be the 190th residue in the first polymer but rather corresponds to the residue found at the 190th position in the reference polymer; those of ordinary skill in the art readily appreciate how to identify “corresponding” amino acids, including through use of one or more commercially-available algorithms specifically designed for polymer sequence comparisons.

“Detection entity”: The term “detection entity” as used herein refers to any element, molecule, functional group, compound, fragment or moiety that is detectable. In some embodiments, a detection entity is provided or utilized alone. In some embodiments, a detection entity is provided and/or utilized in association with (e.g., joined to) another agent. Examples of detection entities include, but are not limited to: various ligands, radionuclides (e.g., ³H, ¹⁴C, ¹⁸F, ¹⁹F, ³²P, ³⁵S, ¹³⁵I, ¹²⁵I, ¹²³I, ⁶⁴Cu, ¹⁸⁷Re, ¹¹¹In, ⁹⁰Y, ^{99m}Tc, ¹⁷⁷Lu, ⁸⁹Zr etc.), fluorescent dyes (for specific exemplary fluorescent dyes, see below), chemiluminescent agents (such as, for example, acridinum esters, stabilized dioxetanes, and the like), bioluminescent agents, spectrally resolvable inorganic fluorescent semiconductors nanocrystals (i.e., quantum dots), metal nanoparticles (e.g., gold, silver, copper, platinum, etc.) nanoclusters, paramagnetic metal ions, enzymes (for specific examples of enzymes, see below), colorimetric labels (such as, for example, dyes, colloidal gold, and the like), biotin, dioxigenin, haptens, and proteins for which antisera or monoclonal antibodies are available.

“Determine”: Many methodologies described herein include a step of “determining”. Those of ordinary skill in the art, reading the present specification, will appreciate that such “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

“Encapsulated”: The term “encapsulated” is used herein to refer to substances that are completely surrounded by another material.

“Functional”: As used herein, a “functional” biological molecule is a biological molecule in a form in which it exhibits a property and/or activity by which it is characterized. A biological molecule may have two functions (i.e., bi-functional) or many functions (i.e., multifunctional).

“High Molecular Weight Polymer”: As used herein, the term “high molecular weight polymer” refers to polymers and/or polymer solutions included of polymers (e.g., protein polymers, such as silk) having molecular weights of at least about 200 kDa, and where no more than 30% of the silk fibroin has a molecular weight of less than 100 kDa. In some embodiments, high molecular weight polymers and/or polymer solutions have an average molecular weight of at least about 100 kDa or more, including, e.g., at least about 150 kDa, at least about 200 kDa, at least about 250 kDa, at least about 300 kDa, at least about 350 kDa or more. In some embodiments, high molecular weight polymers have a molecular weight distribution, no more than 50%, for example, including, no more than 40%, no more than 30%, no more than 20%, no more than 10%, of the silk fibroin can have a molecular weight of less than 150 kDa, or less than 125 kDa, or less than 100 kDa.

“Hydrolytically degradable”: As used herein, the term “hydrolytically degradable” is used to refer to materials that degrade by hydrolytic cleavage. In some embodiments, hydrolytically degradable materials degrade in water. In some embodiments, hydrolytically degradable materials degrade in water in the absence of any other agents or materials. In some embodiments, hydrolytically degradable materials degrade completely by hydrolytic cleavage, e.g., in water. By contrast, the term “non-hydrolytically degradable” typically refers to materials that do not fully degrade by hydrolytic cleavage and/or in the presence of water (e.g., in the sole presence of water).

“Hydrophilic”: As used herein, the term “hydrophilic” and/or “polar” refers to a tendency to mix with, or dissolve easily in, water.

“Hydrophobic”: As used herein, the term “hydrophobic” and/or “non-polar”, refers to a tendency to repel, not combine with, or an inability to dissolve easily in, water.

“Identity”: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

“Low Molecular Weight Polymer”: As used herein, the term “low molecular weight polymer” refers to polymers and/or polymer solutions, such as silk, included of polymers (e.g., protein polymers) having molecular weights within the range of about 20 kDa-about 400 kDa. In some embodiments, low molecular weight polymers (e.g., protein polymers) have molecular weights within a range between a lower bound (e.g., about 20 kDa, about 30 kDa, about 40 kDa, about 50 kDa, about 60 kDa, or more) and an upper bound (e.g., about 400 kDa, about 375 kDa, about 350 kDa, about 325 kDa, about 300 kDa, or less). In some embodiments, low molecular weight polymers (e.g., protein polymers such as silk) are substantially free of, polymers having a molecular weight above about 400 kD. In some embodiments, the highest molecular weight polymers in provided materials are less than about 300-about 400 kD (e.g., less than about 400 kD, less than about 375 kD, less than about 350 kD, less than about 325 kD, less than about 300 kD, etc). In some embodiments, a low molecular weight polymer and/or polymer solution can include a population of polymer fragments having a range of molecular weights, characterized in that: no more than 15% of the total moles of polymer fragments in the population has a molecular weight exceeding 200 kDa, and at least 50% of the total moles of the silk fibroin fragments in the population has a molecular weight within a specified range, where the specified range is between about 3.5 kDa and about 120 kDa or between about 5 kDa and about 125 kDa.

“Marker”: A marker, as used herein, refers to an entity or moiety whose presence or level is a characteristic of a particular state or event. In some embodiments, presence or level of a particular marker may be characteristic of presence or stage of a disease, disorder, or condition. To give but one example, in some embodiments, the term refers to a gene expression product that is characteristic of a particular tumor, tumor subclass, stage of tumor, etc. Alternatively or additionally, in some embodiments, a presence or level of a particular marker correlates with activity (or activity level) of a particular signaling pathway, for example that may be characteristic of a particular class of tumors. The statistical significance of the presence or absence of a marker may vary depending upon the particular marker. In some embodiments, detection of a marker is highly specific in that it reflects a high probability that the tumor is of a particular subclass. Such specificity may come at the cost of sensitivity (i.e., a negative result may occur even if the tumor is a tumor that would be expected to express the marker). Conversely, markers with a high degree of sensitivity may be less specific that those with lower sensitivity. According to the present disclosure a useful marker need not distinguish tumors of a particular subclass with 100% accuracy.

“Modulator”: The term “modulator” is used to refer to an entity whose presence or level in a system in which an activity of interest is observed correlates with a change in level and/or nature of that activity as compared with that observed under otherwise comparable conditions when the modulator is absent. In some embodiments, a modulator is an activator, in that activity is increased in its presence as compared with that observed under otherwise comparable conditions when the modulator is absent. In some embodiments, a modulator is an antagonist or inhibitor, in that activity is reduced in its presence as compared with otherwise comparable conditions when the modulator is absent. In some embodiments, a modulator interacts directly with a target entity whose activity is of interest. In some embodiments, a modulator interacts indirectly (i.e., directly with an intermediate agent that interacts with the target entity) with a target entity whose activity is of interest. In some embodiments, a modulator affects level of a target entity of interest; alternatively or additionally, in some embodiments, a modulator affects activity of a target entity of interest without affecting level of the target entity. In some embodiments, a modulator affects both level and activity of a target entity of interest, so that an observed difference in activity is not entirely explained by or commensurate with an observed difference in level.

“Nanoparticle”: As used herein, the term “nanoparticle” refers to a particle having a diameter of less than 1000 nanometers (nm). In some embodiments, a nanoparticle has a diameter of less than 300 nm, as defined by the National Science Foundation. In some embodiments, a nanoparticle has a diameter of less than 100 nm as defined by the National Institutes of Health. In some embodiments, nanoparticles are micelles in that they include an enclosed compartment, separated from the bulk solution by a micellar membrane, typically included of amphiphilic entities which surround and enclose a space or compartment (e.g., to define a lumen). In some embodiments, a micellar membrane is included of at least one polymer, such as for example a biocompatible and/or biodegradable polymer.

“Nanoparticle composition”: As used herein, the term “nanoparticle composition” refers to a composition that contains at least one nanoparticle and at least one additional agent or ingredient. In some embodiments, a nanoparticle composition contains a substantially uniform collection of nanoparticles as described herein.

“Physiological conditions”: The phrase “physiological conditions”, as used herein, relates to the range of chemical (e.g., pH, ionic strength) and biochemical (e.g., enzyme concentrations) conditions likely to be encountered in the intracellular and extracellular fluids of tissues. For most tissues, the physiological pH ranges from about 6.8 to about 8.0 and a temperature range of about 20-40 degrees Celsius, about 25-40° C., about 30-40° C., about 35-40° C., about 37° C., atmospheric pressure of about 1. In some embodiments, physiological conditions utilize or include an aqueous environment (e.g., water, saline, Ringers solution, or other buffered solution); in some such embodiments, the aqueous environment is or includes a phosphate buffered solution (e.g., phosphate-buffered saline).

“Polypeptide”: The term “polypeptide”, as used herein, generally has its art-recognized meaning of a polymer of at least three amino acids, linked to one another by peptide bonds. In some embodiments, the term is used to refer to specific functional classes of polypeptides. For each such class, the present specification provides several examples of amino acid sequences of known exemplary polypeptides within the class; in some embodiments, such known polypeptides are reference polypeptides for the class. In such embodiments, the term

“polypeptide” refers to any member of the class that shows significant sequence homology or identity with a relevant reference polypeptide. In many embodiments, such member also shares significant activity with the reference polypeptide. Alternatively or additionally, in many embodiments, such member also shares a particular characteristic sequence element with the reference polypeptide (and/or with other polypeptides within the class; in some embodiments with all polypeptides within the class). For example, in some embodiments, a member polypeptide shows an overall degree of sequence homology or identity with a reference polypeptide that is at least about 30-40%, and is often greater than about 50%, 60%, 70%, 80%, 90%, 91%, 92%, 93%, 94%, 95%, 96%, 97%, 98%, 99% or more and/or includes at least one region (i.e., a conserved region that may in some embodiments may be or include a characteristic sequence element) that shows very high sequence identity, often greater than 90% or even 95%, 96%, 97%, 98%, or 99%. Such a conserved region usually encompasses at least 3-4 and often up to 20 or more amino acids; in some embodiments, a conserved region encompasses at least one stretch of at least 2, 3, 4, 5, 6, 7, 8, 9, 10, 11, 12, 13, 14, 15 or more contiguous amino acids. In some embodiments, a useful polypeptide may include or consist of a fragment of a parent polypeptide. In some embodiments, a useful polypeptide as may include or consist of a plurality of fragments, each of which is found in the same parent polypeptide in a different spatial arrangement relative to one another than is found in the polypeptide of interest (e.g., fragments that are directly linked in the parent may be spatially separated in the polypeptide of interest or vice versa, and/or fragments may be present in a different order in the polypeptide of interest than in the parent), so that the polypeptide of interest is a derivative of its parent polypeptide. In some embodiments, a polypeptide may include natural amino acids, non-natural amino acids, or both. In some embodiments, a polypeptide may include only natural amino acids or only non-natural amino acids. In some embodiments, a polypeptide may include D-amino acids, L-amino acids, or both. In some embodiments, a polypeptide may include only D-amino acids. In some embodiments, a polypeptide may include only L-amino acids. In some embodiments, a polypeptide may include one or more pendant groups, e.g., modifying or attached to one or more amino acid side chains, and/or at the polypeptide's N-terminus, the polypeptide's C-terminus, or both. In some embodiments, a polypeptide may be cyclic. In some embodiments, a polypeptide is not cyclic. In some embodiments, a polypeptide is linear.

“Porosity”: The term “porosity” as used herein, refers to a measure of void spaces in a material and is a fraction of volume of voids over the total volume, as a percentage between 0 and 100%. A determination of a porosity is known to a skilled artisan using standardized techniques, for example mercury porosimetry and gas adsorption (e.g., nitrogen adsorption).

“Protein”: As used herein, the term “protein” refers to a polypeptide (i.e., a string of at least two amino acids linked to one another by peptide bonds). Proteins may include moieties other than amino acids (e.g., may be glycoproteins, proteoglycans, etc.) and/or may be otherwise processed or modified. Those of ordinary skill in the art will appreciate that a “protein” can be a complete polypeptide chain as produced by a cell (with or without a signal sequence), or can be a characteristic portion thereof. Those of ordinary skill will appreciate that a protein can sometimes include more than one polypeptide chain, for example linked by one or more disulfide bonds or associated by other means. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may include natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

“Reference”: The term “reference” is often used herein to describe a standard or control agent, individual, population, sample, sequence or value against which an agent, individual, population, sample, sequence or value of interest is compared. In some embodiments, a reference agent, individual, population, sample, sequence or value is tested and/or determined substantially simultaneously with the testing or determination of the agent, individual, population, sample, sequence or value of interest. In some embodiments, a reference agent, individual, population, sample, sequence or value is a historical reference, optionally embodied in a tangible medium. Typically, as would be understood by those skilled in the art, a reference agent, individual, population, sample, sequence or value is determined or characterized under conditions comparable to those utilized to determine or characterize the agent, individual, population, sample, sequence or value of interest.

“Solution”: As used herein, the term “solution” broadly refers to a homogeneous mixture composed of one phase. Typically, a solution includes a solute or solutes dissolved in a solvent or solvents. It is characterized in that the properties of the mixture (such as concentration, temperature, and density) can be uniformly distributed through the volume. In the context of the present application, therefore, a “silk fibroin solution” refers to silk fibroin protein in a soluble form, dissolved in a solvent, such as water. In some embodiments, silk fibroin solutions may be prepared from a solid-state silk fibroin material (i.e., silk matrices), such as silk films and other scaffolds. Typically, a solid-state silk fibroin material is reconstituted with an aqueous solution, such as water and a buffer, into a silk fibroin solution. It should be noted that liquid mixtures that are not homogeneous, e.g., colloids, suspensions, emulsions, are not considered solutions.

“Stable”: The term “stable,” when applied to compositions herein, means that the compositions maintain one or more aspects of their physical structure and/or activity over a period of time under a designated set of conditions. In some embodiments, the period of time is at least about one hour; in some embodiments, the period of time is about 5 hours, about 10 hours, about one (1) day, about one (1) week, about two (2) weeks, about one (1) month, about two (2) months, about three (3) months, about four (4) months, about five (5) months, about six (6) months, about eight (8) months, about ten (10) months, about twelve (12) months, about twenty-four (24) months, about thirty-six (36) months, or longer. In some embodiments, the period of time is within the range of about one (1) day to about twenty-four (24) months, about two (2) weeks to about twelve (12) months, about two (2) months to about five (5) months, etc. In some embodiments, the designated conditions are ambient conditions (e.g., at room temperature and ambient pressure). In some embodiments, the designated conditions are physiologic conditions (e.g., in vivo or at about 37° C. for example in serum or in phosphate buffered saline). In some embodiments, the designated conditions are under cold storage (e.g., at or below about 4° C., −20° C., or −70° C.). In some embodiments, the designated conditions are in the dark.

“Substantially”: As used herein, the term “substantially”, and grammatic equivalents, refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result.

“Variant”: As used herein, the term “variant” refers to an entity that shows significant structural identity with a reference entity but differs structurally from the reference entity in the presence or level of one or more chemical moieties as compared with the reference entity. In many embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a small molecule may have a characteristic core structural element (e.g., a macrocycle core) and/or one or more characteristic pendent moieties so that a variant of the small molecule is one that shares the core structural element and the characteristic pendent moieties but differs in other pendent moieties and/or in types of bonds present (single vs double, E vs Z, etc.) within the core, a polypeptide may have a characteristic sequence element included of a plurality of amino acids having designated positions relative to one another in linear or three-dimensional space and/or contributing to a particular biological function, a nucleic acid may have a characteristic sequence element included of a plurality of nucleotide residues having designated positions relative to on another in linear or three-dimensional space. For example, a variant polypeptide may differ from a reference polypeptide as a result of one or more differences in amino acid sequence and/or one or more differences in chemical moieties (e.g., carbohydrates, lipids, etc.) covalently attached to the polypeptide backbone. In some embodiments, a variant polypeptide shows an overall sequence identity with a reference polypeptide that is at least 85%, 86%, 87%, 88%, 89%, 90%, 91%, 92%, 93%, 94%, 95%, 96%, 97%, or 99%. Alternatively or additionally, in some embodiments, a variant polypeptide does not share at least one characteristic sequence element with a reference polypeptide. In some embodiments, the reference polypeptide has one or more biological activities. In some embodiments, a variant polypeptide shares one or more of the biological activities of the reference polypeptide. In some embodiments, a variant polypeptide lacks one or more of the biological activities of the reference polypeptide. In some embodiments, a variant polypeptide shows a reduced level of one or more biological activities as compared with the reference polypeptide. In many embodiments, a polypeptide of interest is considered to be a “variant” of a parent or reference polypeptide if the polypeptide of interest has an amino acid sequence that is identical to that of the parent but for a small number of sequence alterations at particular positions. Typically, fewer than 20%, 15%, 10%, 9%, 8%, 7%, 6%, 5%, 4%, 3%, 2% of the residues in the variant are substituted as compared with the parent. In some embodiments, a variant has 10, 9, 8, 7, 6, 5, 4, 3, 2, or 1 substituted residue as compared with a parent. Often, a variant has a very small number (e.g., fewer than 5, 4, 3, 2, or 1) number of substituted functional residues (i.e., residues that participate in a particular biological activity). Furthermore, a variant typically has not more than 5, 4, 3, 2, or 1 additions or deletions, and often has no additions or deletions, as compared with the parent. Moreover, any additions or deletions are typically fewer than about 25, about 20, about 19, about 18, about 17, about 16, about 15, about 14, about 13, about 10, about 9, about 8, about 7, about 6, and commonly are fewer than about 5, about 4, about 3, or about 2 residues. In some embodiments, the parent or reference polypeptide is one found in nature. As will be understood by those of ordinary skill in the art, a plurality of variants of a particular polypeptide of interest may commonly be found in nature, particularly when the polypeptide of interest is an infectious agent polypeptide.

### DETAILED DESCRIPTION OF CERTAIN EMBODIMENTS

Among other things, the present disclosure provides systems useful for preparing silk fibroin solutions and methods of preparing such silk fibroin solutions. Various embodiments according to the present disclosure are described in detail herein. In particular, in some embodiments, the present disclosure describes systems for purifying and concentrating silk fibroin solutions and methods for preparing such silk fibroin solutions.

Pure concentrated silk fibroin solutions are further processed to form materials useful in various applications, including, for example: biomaterials, biomedical devices, biosensing, controlled release applications, drug delivery, electronics, materials for tunable degradation, optics, photonics, regenerative medicine, sensors, textiles, tissue engineering applications, tissue regeneration, tissue scaffolding, and/or wound clotting.

Using traditional protein purification apparatus and methods, transforming silk to into useable solutions of silk fibroin is both labor and time intensive. Automation of certain steps of prior processes has shown to reduce time; but a consequence of this automation is increased handling due to faster processing speeds. Speed induced handling in combination with silk protein's sensitivity to shear results in transformation of silk structures from random coil to higher order configurations, such a beta sheet. Aggregation of silk proteins in these higher order configurations is less useful for silk fibroin solution applications. Moreover, silk protein aggregation forms a thicker more viscous silk fibroin solution that clogs the membranes that work to process the silk thereby inadvertently slowing the process.

In some embodiments, the present disclosure relates to systems and methods for automated preparation of purified and concentrated silk fibroin solutions in high volume over a short time with minimal aggregation and useful in the production of silk fibroin materials such as, fibers, films, foams, hydrogels matrices, scaffolds, etc.

### Silk Processing

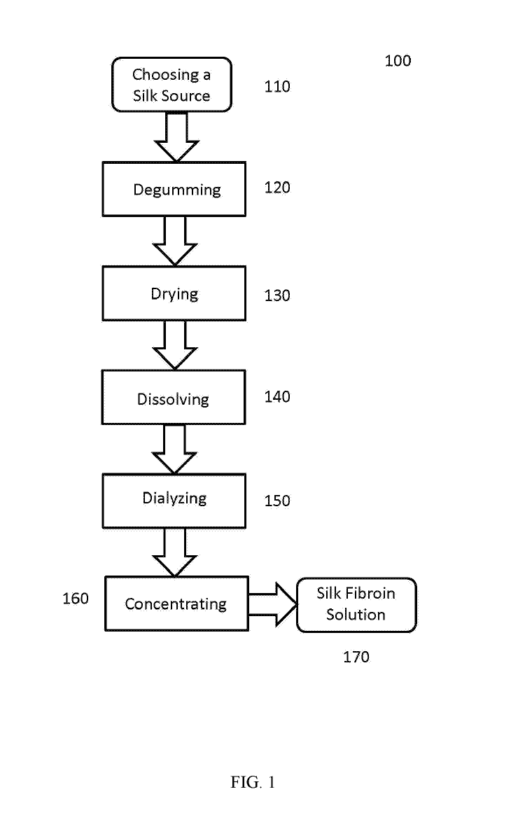

In some embodiments, silk solutions can be prepared by any conventional method known to one skilled in the art. For example, methods of processing silk are disclosed for example in WO 2005/012606, WO 2014/011644, and WO 2014/145002, which are each hereby incorporated by reference in their entirety herein. In some embodiments, silk solution processing is described in stages as shown in FIG. 1. In some embodiments, silk solution processing, 100 generally is defined in six stages including choosing a silk source 110; degumming 120, drying 130, dissolving 140, dialyzing 150, and concentrating 160.

### Choosing a Silk Source

In some embodiments, a first step includes choosing a silk source 110. In some embodiments, silk is a natural protein fiber produced in a specialized gland of certain organisms. Silk production is in organisms is especially common in silkworms. Silk production is also common in Hymenoptera (bees, wasps, and ants), and is sometimes used in nest construction. Other types of arthropod also produce silk, most notably various arachnids such as spiders (e.g., spider silk). Silk fibers generated by insects and spiders represent the strongest natural fibers known and rival even synthetic high performance fibers.

Silk has been a highly desired and widely used textile since its first appearance in ancient China (see Elisseeff, “The Silk Roads: Highways of Culture and Commerce,” Berghahn Books/UNESCO, New York (2000); see also Vainker, “Chinese Silk: A Cultural History,” Rutgers University Press, Piscataway, N.J. (2004)).

Glossy and smooth, silk is favored by not only fashion designers but also tissue engineers because it is mechanically tough but degrades harmlessly inside the body, offering new opportunities as a highly robust and biocompatible material substrate (see Altman et al., Biomaterials, 24: 401 (2003); see also Sashina et al., Russ. J. Appl. Chem., 79: 869 (2006)).

The unique mechanical properties of reprocessed silk such as fibroin and its biocompatibility make the silk fibers especially attractive for use in biotechnological materials and medical applications. Silk provides an important set of material options for biomaterials and tissue engineering because of the impressive mechanical properties, biocompatibility and biodegradability (see Altman, G. H., et al., Biomaterials 2003, 24, 401-416; Cappello, J., et al., J. Control. Release 1998, 53, 105-117; Foo, C. W. P., et al., Adv. Drug Deliver. Rev. 2002, 54, 1131-1143; Dinerman, A. A., et al., J. Control. Release 2002, 82, 277-287; Megeed, Z., et al., Adv. Drug Deliver. Rev. 2002, 54, 1075-1091; Petrini, P., et al., J. Mater. Sci-Mater. M 2001, 12, 849-853; Altman, G. H., et al., Biomaterials 2002, 23, 4131-4141; Panilaitis, B., et al., Biomaterials 2003, 24, 3079-3085). For example, 3-dimensional porous silk scaffolds have been described for use in tissue engineering (Meinel et al., Ann Biomed Eng. 2004 January; 32(1):112-22; Nazarov, R., et al., Biomacromolecules in press). Further, regenerated silk fibroin films have been explored as oxygen- and drug-permeable membranes, supports for enzyme immobilization, and substrates for cell culture (Minoura, N., et al., Polymer 1990, 31, 265-269; Chen, J., et al., Minoura, N., Tanioka, A. 1994, 35, 2853-2856; Tsukada, M., et al., Polym. Sci. Part B Polym. Physics 1994, 32, 961-968). In addition, silk hydrogels have found numerous applications in tissue engineering, as well as in drug delivery (Megeed et al., Pharm Res. 2002 July; 19(7):954-9; Dinerman et al., J Control Release. 2002 Aug. 21; 82(2-3):277-87).

Silk is naturally produced by various species, including, without limitation: Antheraea mylitta; Antheraea pernyi; Antheraea yamamai; Galleria mellonella; Bombyx mori; Bombyx mandarina; Galleria mellonella; Nephila clavipes; Nephila senegalensis; Gasteracantha mammosa; Argiope aurantia; Araneus diadematus; Latrodectus geometricus; Araneus bicentenarius; Tetragnatha versicolor; Araneus ventricosus; Dolomedes tenebrosus; Euagrus chisoseus; Plectreurys tristis; Argiope trifasciata; and Nephila madagascariensis.

As is known in the art, silks are modular in design, with large internal repeats flanked by shorter (˜100 amino acid) terminal domains (N and C termini). Naturally-occurring silks have high molecular weight (200 to 350 kDa or higher) with transcripts of 10,000 base pairs and higher and >3000 amino acids (reviewed in Omenetto and Kaplan (2010) Science 329: 528-531). The larger modular domains are interrupted with relatively short spacers with hydrophobic charge groups in the case of silkworm silk. N- and C-termini are involved in the assembly and processing of silks, including pH control of assembly. The N- and C-termini are highly conserved, in spite of their relatively small size compared with the internal modules. Table 1, below, provides an exemplary list of silk-producing species and silk proteins:

TABLE 1
An exemplary list of silk-producing species and silk proteins
(adopted from Bini et al. (2003), J. Mol. Biol. 335(1): 27-40).

| | | | |
|---|---|---|---|
| Accession | Species | Producing gland | Protein |

Silkworms

| | | | |
|---|---|---|---|
| AAN28165 | Antheraea mylitta | Salivary | Fibroin |
| AAC32606 | Antheraea pernyi | Salivary | Fibroin |
| AAK83145 | Antheraea yamamai | Salivary | Fibroin |
| AAG10393 | Galleria mellonella | Salivary | Heavy-chain fibroin |
| | | | (N-terminal) |
| AAG10394 | Galleria mellonella | Salivary | Heavy-chain fibroin |
| | | | (C-terminal) |
| P05790 | Bombyx mori | Salivary | Fibroin heavy chain |
| | | | precursor, Fib-H, H-fibroin |
| CAA27612 | Bombyx mandarina | Salivary | Fibroin |
| Q26427 | Galleria mellonella | Salivary | Fibroin light chain precursor, |
| | | | Fib-L, L-fibroin, PG-1 |
| P21828 | Bombyx mori | Salivary | Fibroin light chain precursor, |
| | | | Fib-L, L-fibroin |

Spiders

| | | | |
|---|---|---|---|
| P19837 | Nephila clavipes | Major ampullate | Spidroin 1, |
| | | | dragline silk fibroin 1 |
| P46804 | Nephila clavipes | Major ampullate | Spidroin 2, |
| | | | dragline silk fibroin 2 |
| AAK30609 | Nephila senegalensis | Major ampullate | Spidroin 2 |
| AAK30601 | Gasteracantha | Major ampullate | Spidroin 2 |
| | mammosa | | |
| AAK30592 | Argiope aurantia | Major ampullate | Spidroin 2 |
| AAC47011 | Araneus diadematus | Major ampullate | Fibroin-4, ADF-4 |
| AAK30604 | Latrodectus | Major ampullate | Spidroin 2 |
| | geometricus | | |
| AAC04503 | Araneus bicentenarius | Major ampullate | Spidroin 2 |
| AAK30615 | Tetragnatha versicolor | Major ampullate | Spidroin 1 |
| AAN85280 | Araneus ventricosus | Major ampullate | Dragline silk protein-1 |
| AAN85281 | Araneus ventricosus | Major ampullate | Dragline silk protein-2 |
| AAC14589 | Nephila clavipes | Minor ampullate | MiSp1 silk protein |
| AAK30598 | Dolomedes tenebrosus | Ampullate | Fibroin 1 |
| AAK30599 | Dolomedes tenebrosus | Ampullate | Fibroin 2 |
| AAK30600 | Euagrus chisoseus | Combined | Fibroin 1 |
| AAK30610 | Plectreurys tristis | Larger ampule- | Fibroin 1 |
| | | shaped | |
| AAK30611 | Plectreurys tristis | Larger ampule- | Fibroin 2 |
| | | shaped | |
| AAK30612 | Plectreurys tristis | Larger ampule- | Fibroin 3 |
| | | shaped | |
| AAK30613 | Plectreurys tristis | Larger ampule- | Fibroin 4 |
| | | shaped | |
| AAK30593 | Argiope trifasciata | Flagelliform | Silk protein |
| AAF36091 | Nephila | Flagelliform | Fibroin, silk protein |
| | madagascariensis | | (N-terminal) |
| AAF36092 | Nephila | Flagelliform | Silk protein |
| | madagascariensis | | (C-terminal) |
| AAC38846 | Nephila clavipes | Flagelliform | Fibroin, silk protein |
| | | | (N-terminal) |
| AAC38847 | Nephila clavipes | Flagelliform | Silk protein |
| | | | (C-terminal) |

In general, silk for use in accordance with the present disclosure may be produced by any such organism, from a recombinant source or may be prepared through an artificial process, for example, involving genetic engineering of cells or organisms to produce a silk protein and/or chemical synthesis. In some embodiments of the present disclosure, silk is produced by the silkworm, Bombyx mori.

In some embodiments, a silk source is a silkworm cocoon. In some embodiments, a silk source is a bave silk, which has been unreeled from silkworm cocoons by a supplier and spun together to form a continuous spool.

### Degumming

Generally suppliers provide silk material in its raw form, with a glue-like protein, sericin, coating the underlying silk fibroin protein. Many applications of silk require that the sericin be removed. The process of removing sericin known as degumming 120.

As used herein, the term “silk fibroin” refers to silk fibroin protein, whether produced by silkworm, spider, or other insect, or otherwise generated (Lucas et al., 13 Adv. Protein Chem., 107-242 (1958)). In some embodiments, silk fibroin is obtained from a solution containing a dissolved silkworm silk or spider silk. For example, in some embodiments, silkworm silk fibroins are obtained, from the cocoon of Bombyx mori. In some embodiments, spider silk fibroins are obtained, for example, from Nephila clavipes. In the alternative, in some embodiments, silk fibroins suitable for use in the invention are obtained from a solution containing a genetically engineered silk harvested from bacteria, yeast, mammalian cells, transgenic animals or transgenic plants. See, e.g., WO 97/08315 and U.S. Pat. No. 5,245,012, each of which is incorporated herein as reference in its entirety.

Fibroin is a type of structural protein produced by certain spider and insect species that produce silk. Cocoon silk produced by the silkworm, Bombyx mori, is of particular interest because it offers low-cost, bulk-scale production suitable for a number of commercial applications, such as textile.

Silkworm cocoon silk contains two structural proteins, the fibroin heavy chain (˜350 kDa) and the fibroin light chain (˜25 kDa), which are associated with a family of non-structural proteins termed sericin, which glue the fibroin brings together in forming the cocoon. The heavy and light chains of fibroin are linked by a disulfide bond at the C-terminus of the two subunits (see Takei, F., Kikuchi, Y., Kikuchi, A., Mizuno, S. and Shimura, K. (1987) 105 J. Cell Biol., 175-180; see also Tanaka, K., Mori, K. and Mizuno, S. 114 J. Biochem. (Tokyo), 1-4 (1993); Tanaka, K., Kajiyama, N., Ishikura, K., Waga, S., Kikuchi, A., Ohtomo, K., Takagi, T. and Mizuno, S., 1432 Biochim. Biophys. Acta., 92-103 (1999); Y Kikuchi, K Mori, S Suzuki, K Yamaguchi and S Mizuno, “Structure of the Bombyx mori fibroin light-chain-encoding gene: upstream sequence elements common to the light and heavy chain,” 110 Gene, 151-158 (1992)). The sericins are a high molecular weight, soluble glycoprotein constituent of silk which gives the stickiness to the material. These glycoproteins are hydrophilic and can be easily removed from cocoons by boiling in water.

In some embodiments, silk solutions of the present disclosure contain fibroin proteins, essentially free of sericins. In some embodiments, silk solutions used to fabricate various compositions of the present disclosure contain both heavy and light chains of fibroin, but are essentially free of other proteins. In some embodiments, heavy chain and light chain silk fibroin are linked. In some embodiments, heavy and light chain silk fibroin are linked via at least one disulfide bond. In some embodiments where the heavy and light chains of fibroin are present, they are linked via one, two, three or more disulfide bonds.

Although different species of silk-producing organisms and different types of silk have different amino acid compositions, various silk fibroin proteins share certain structural features. A general trend in silk fibroin structure is a sequence of amino acids that is characterized by usually alternating glycine and alanine, or alanine alone. These “Alanine-rich” hydrophobic blocks are typically separated by segments of amino acids with bulky side-groups (e.g., hydrophilic spacers). Such configuration allows fibroin molecules to self-assemble into a beta-sheet conformation.

In some embodiments, raw silk fibroin that is free of sericin is produced by degumming 120. In some embodiments, degumming is achieved by first cutting or chopping silk into small pieces. In some embodiments, silk pieces are chopped and/or cut so that pieces are massed at about 5 g. In some embodiments, a size for silk pieces will vary. In some embodiments, a size for silk pieces is generally uniform. In some embodiments, silk pieces are massed between about 0.5 g and 10 g.

In some embodiments, small pieces of silk are then soaked in boiling water containing a detergent, such as for example 0.02 M Na₂CO₃. In some embodiments, a boiling detergent solution contains for example between about 1 gram and 10 grams of a detergent dissolved in a solvent. In some embodiments, for example a boiling detergent solution includes between about 1 L and 5 L of a solvent, such as water.

In some embodiments, degumming time is about 30 minutes. In some embodiments, polymers of silk fibroin fragments can be derived by degumming silk cocoons at or close to (e.g., within 5% around) an atmospheric boiling temperature for at least about: 1 minute of boiling, 2 minutes of boiling, 3 minutes of boiling, 4 minutes of boiling, 5 minutes of boiling, 6 minutes of boiling, 7 minutes of boiling, 8 minutes of boiling, 9 minutes of boiling, 10 minutes of boiling, 11 minutes of boiling, 12 minutes of boiling, 13 minutes of boiling, 14 minutes of boiling, 15 minutes of boiling, 16 minutes of boiling, 17 minutes of boiling, 18 minutes of boiling, 19 minutes of boiling, 20 minutes of boiling, 25 minutes of boiling, 30 minutes of boiling, 35 minutes of boiling, 40 minutes of boiling, 45 minutes of boiling, 50 minutes of boiling, 55 minutes of boiling, 60 minutes or longer, including, e.g., at least 70 minutes, at least 80 minutes, at least 90 minutes, at least 100 minutes, at least 110 minutes, at least about 120 minutes or longer. As used herein, the term “atmospheric boiling temperature” refers to a temperature at which a liquid boils under atmospheric pressure.

In some embodiments, silk fibroin solutions of the present disclosure produced from silk fibroin fragments can be formed by degumming silk cocoons in an aqueous solution at temperatures of: about 30° C., about 35° C., about 40° C., about 45° C., about 50° C., about 45° C., about 60° C., about 65° C., about 70° C., about 75° C., about 80° C., about 85° C., about 90° C., about 95° C., about 100° C., about 105° C., about 110° C., about 115° C., about at least 120° C.

In some embodiments, such elevated temperature can be achieved by carrying out at least portion of the heating process (e.g., boiling process) under pressure. For example, suitable pressure under which silk fibroin fragments described herein can be produced are typically between about 10-40 psi, e.g., about 11 psi, about 12 psi, about 13 psi, about 14 psi, about 15 psi, about 16 psi, about 17 psi, about 18 psi, about 19 psi, about 20 psi, about 21 psi, about 22 psi, about 23 psi, about 24 psi, about 25 psi, about 26 psi, about 27 psi, about 28 psi, about 29 psi, about 30 psi, about 31 psi, about 32 psi, about 33 psi, about 34 psi, about 35 psi, about 36 psi, about 37 psi, about 38 psi, about 39 psi, or about 40 psi.

In some embodiments, silk fibroin solutions include silk fibroin fragments derived from silk fibroin protein or variants thereof. In some embodiments, the present disclosure provides silk fibroin fragments which are generally silk fibroin peptide chains or polypeptides that are smaller than naturally occurring full length silk fibroin counterpart, such that one or more of the silk fibroin fragments within a population or composition. In some embodiments, for example, silk fibroin solutions include silk fibroin polypeptides having an average molecular weight of between about 1 kDa and about 400 kDa. In some embodiments, suitable ranges of silk fibroin solutions include, but are not limited to: silk fibroin polypeptides having an average molecular weight of between about 3.5 kDa and about 200 kDa; silk fibroin polypeptides having an average molecular weight of between about 3.5 kDa and about 150 kDa; silk fibroin polypeptides having an average molecular weight of between about 3.5 kDa and about 120 kDa. In some embodiments, silk fibroin polypeptides have an average molecular weight of: about 3.5 kDa, about 4 kDa, about 4.5 kDa, about 5 kDa, about 6 kDa, about 7 kDa, about 8 kDa, about 9 kDa, about 10 kDa, about 15 kDa, about 20 kDa, about 25 kDa, about 30 kDa, about 35 kDa, about 40 kDa, about 45 kDa, about 50 kDa, about 55 kDa, about 60 kDa, about 65 kDa, about 70 kDa, about 75 kDa, about 80 kDa, about 85 kDa, about 90 kDa, about 95 kDa, about 100 kDa, about 105 kDa, about 110 kDa, about 115 kDa, about 120 kDa, about 125 kDa, about 150 kDa, about 200 kDa, about 250 kDa, about 300 kDa, about 350 kDa, or about 400 kDa.

In some embodiments, silk fibroin solutions are or include silk fibroin and/or silk fibroin fragments. In some embodiments, silk fibroin and/or silk fibroin fragments of various molecular weights may be used. In some embodiments, silk fibroin and/or silk fibroin fragments of various molecular weights are silk fibroin polypeptides. In some embodiments, silk fibroin polypeptides are “reduced”, for instance, smaller than the original or wild type counterpart, may be referred to as “low molecular weight silk fibroin”. For more details related to low molecular weight silk fibroins, see international application PCT/US2014/029636, published as WO 2014/145002 on Sep. 18, 2014, entitled “LOW MOLECULAR WEIGHT SILK COMPOSITIONS AND STABILIZING SILK COMPOSITIONS,” the entire contents of which are incorporated herein by reference.

In some embodiments, silk fibroin solutions are or include silk fibroin and/or silk fibroin fragments. In some embodiments, silk fibroin and/or silk fibroin fragments of various molecular weights may be used. In some embodiments, silk fibroin and/or silk fibroin fragments of various molecular weights are silk fibroin polypeptides. In some embodiments, silk fibroin polypeptides are “larger” and may be referred to as “high molecular weight silk fibroin.” For more details related to high molecular weight silk fibroins, see: international application PCT/US2013/049740, published as WO2014/011644 on Jan. 16, 2014, entitled “HIGH MOLECULAR WEIGHT SILK FIBROIN AND USES THEREOF,” the entire contents of which are incorporated herein by reference.

### Drying

In some embodiments, after degumming, a silk fibroin solution is placed in a water rinse for a time with occasional stirring and rinsing water is occasionally changed. In some embodiments, silk is rinsed, for example, with water to extract the sericin proteins. In some embodiments, a step of drying 130 follows degumming 120. In some embodiments, silk is dried for example when squeezed out and/or placed in a hood to air dry. In some embodiments, silk fibroin dries for between at least about 2 hours to about 24 hours.

### Dissolving

In some embodiments, silk fibroin solution processing includes dissolving 140 extracted dried silk fibroin. In some embodiments, an extracted and dried silk fibroin is dissolved to form a solution. In some embodiments, a solution is in an aqueous salt solution. In some embodiments, salts useful for this purpose include, salts (typically high ionic strength aqueous salt solutions) and solvents that are known to be used in processing of silk, such as lithium thiocyanate (LiSCN), sodium thiocyanate (NaSCN), calcium thiocynanate (Ca(SCN)₂), magnesium thiocyanate (Mg(SCN)₂), calcium chloride (CaCl₂), calcium nitrate (Ca(NO₃)₂), lithium bromide (LiBr), zinc chloride (ZnCl₂), magnesium chloride (MgCl₂), and copper salts. Other useful salts include those described in U.S. Pat. No. 5,252,285 and/or Sashina et al., “Structure and Solubility of Natural Silk Fibroin,” 79 Russian Journal of Applied Chemistry 6, 869-876 (2006), each of which is hereby incorporated by reference in its entirety herein. To one of skill in the art, dialysis is a known process for preparing silk fibroin solutions and as such the systems and methods of the present disclosure are applicable with other salts or other chemicals capable of solubilizing silk.

In some embodiments, extracted silk is dissolved in lithium bromide. In some embodiments, extracted silk is dissolved in between about 7 M and 13 M LiBr solution. In some embodiments, such a silk fibroin solution is heated. In some embodiments, a silk fibroin solution is heated to about 60° C. In some embodiments, a silk fibroin solution is heated for about 4 hours.

In some embodiments, a dissolved silk fibroin solution has a viscosity of between about 1 cP and about 30 cP. In some embodiments, a dissolved silk fibroin solution has a viscosity of between about 2 cP and about 20 cP. In some embodiments, a dissolved silk fibroin solution has a viscosity of between about 3 cP and about 8 cP. In some embodiments, a dissolved silk fibroin solution has a viscosity of about 1 cP, about 1.5 cP, about 2 cP, about 2.5 cP, about 3 cP, about 3.5 cP, about 4 cP, about 4.5 cP, about 5 cP, about 5.5 cP, about 6 cP, about 6.5 cP, about 7 cP, about 7.5 cP, about 8 cP, about 8.5 cP, about 9 cP, about 10 cP, about 11 cP, about 12 cP, about 13 cP, about 14 cP, about 15 cP, about 16 cP, about 17 cP, about 18 cP, about 19 cP, about 20 cP, about 21 cP, about 22 cP, about 23 cP, about 24 cP, about 25 cP, about 26 cP, about 27 cP, about 28 cP, about 29 cP, or about 30 cP.

### Dialyzing

In some embodiments, salts used to dissolve silk fibroin are removed from a dissolved silk fibroin solution using, for example, a dialyzing step 150. and other contaminants or impurities

In some embodiments, a dissolved silk fibroin solution is dialyzed against a solvent. In some embodiments, a dialyzing solvent is water. In some embodiments, a dialyzing solvent is a hyproscopic polymer. In some embodiments, a hyproscopic polymer, for example, is polyethylene glycol (PEG) or amylase. In some embodiments, a hygroscopic polymer is polyethylene glycol (PEG) with a molecular weight of 8,000 to 10,000 g/mol. In some embodiments, PEG has a concentration of 25-50%. In some embodiments, dialyzing a solution against a hygroscopic polymer is also sufficient to control water content in the formation of silk hydrogels.

### Concentrating

In some embodiments, silk fibroin is processed to a concentrated solution prior to processing for textile, medical, mechanical, etc. applications. In some embodiments, a purified concentrated solution is between about <1 wt % and about 30 wt %. In some embodiments, increasing the concentration of the aqueous silk fibroin solution to at least 10 wt % is desirable. In some embodiments, dialysis is performed on a silk fibroin solution for a sufficient time to result in a silk fibroin solution of between 10% and 30 wt %, or greater. In some embodiments, higher concentration allows for the formation of structures, such as, for example, fibers, films, foams, matrices, three-dimensional scaffolds, etc. In some embodiments, purified silk fibroin solutions undergo a concentrating step 160.

### Silk Fibroin Solutions

In some embodiments, silk fibroin solutions 170 are or include silk at any of a variety of concentrations. In some embodiments, silk fibroin may be present in a solution at any weight percentage or concentration suited to the need. In many embodiments, a silk fibroin solution as described and/or utilized herein is an aqueous solution (i.e., includes silk fibroin dissolved in an aqueous solvent such as, for example, water).

In some embodiments, a silk fibroin solution can have silk fibroin at a concentration within a range of about 0.1 mg/mL to about 50 mg/mL. In some embodiments, a silk fibroin solution can include silk fibroin at a concentration of less than about 1 mg/mL, less than about 1.5 mg/mL, less than about 2 mg/mL, less than about 2.5 mg/mL, less than about 3 mg/mL, less than about 3.5 mg/mL, less than about 4 mg/mL, less than about 4.5 mg/mL, less than about 5 mg/mL, less than about 5.5 mg/mL, less than about 6 mg/mL, less than about 6.5 mg/mL, less than about 7 mg/mL, less than about 7.5 mg/mL, less than about 8 mg/mL, less than about 8.5 mg/mL, less than about 9 mg/mL, less than about 9.5 mg/mL, less than about 10 mg/mL, less than about 11 mg/mL, less than about 12 mg/mL, less than about 13 mg/mL, less than about 14 mg/mL, less than about 15 mg/mL, less than about 16 mg/mL, less than about 17 mg/mL, less than about 18 mg/mL, less than about 19 mg/mL, less than about 20 mg/mL, less than about 25 mg/mL, less than about 30 mg/mL, less than about 35 mg/mL, less than about 40 mg/mL, less than about 45 mg/mL, or less than about 50 mg/mL.

In some embodiments, a silk fibroin solution can have silk fibroin at a concentration of about 0.1 wt % to about 95 wt %, 0.1 wt % to about 75 wt %, or 0.1 wt % to about 50 wt %. In some embodiments, a silk fibroin solution can have silk fibroin at a concentration of about 0.1 wt % to about 10 wt %, about 0.1 wt % to about 5 wt %, about 0.1 wt % to about 2 wt %, or about 0.1 wt % to about 1 wt %. In some embodiments, a silk fibroin solution have silk fibroin at a concentration of about 10 wt % to about 50 wt %, about 20 wt % to about 50 wt %, about 25 wt % to about 50 wt %, or about 30 wt % to about 50 wt %. In some embodiments, a weight percent of silk in solution is less than about 1 wt %, is less than about 1.5 wt %, is less than about 2 wt %, is less than about 2.5 wt %, is less than about 3 wt %, is less than about 3.5 wt %, is less than about 4 wt %, is less than about 4.5 wt %, is less than about 5 wt %, is less than about 5.5 wt %, is less than about 6 wt %, is less than about 6.5 wt %, is less than about 7 wt %, is less than about 7.5 wt %, is less than about 8 wt %, is less than about 8.5 wt %, is less than about 9 wt %, is less than about 9.5 wt %, is less than about 10 wt %, is less than about 11 wt %, is less than about 12 wt %, is less than about 13 wt %, is less than about 14 wt %, is less than about 15 wt %, is less than about 16 wt %, is less than about 17 wt %, is less than about 18 wt %, is less than about 19 wt %, is less than about 20 wt %, is less than about 25 wt %, or is less than about 30 wt %.

### Silk Fibroin Solution Purification Systems

Generally, dialysis of dissolved silk fibroin has traditionally involved the use of a Thermo Scientific Slide-A-Lyzer dialysis cassettes (3.5 K molecular weight cut-off). These cassettes have a cellulose membrane that retains proteins larger than 3500 Da while allowing removal of buffer salts and small contaminants. The cassette is first rinsed in distilled water for 30 minutes to soften the membrane. Approximately 12 mL of the silk/LiBr solution is inserted in a 3 to 12 ml cassette and dialyzed. Insertion of the silk solution typically required injecting the solution using a syringe. About 6 water changes are prescribed. The final steps in the process are centrifugation to assist in particle/dirt material from the solution and concentrating, which increases the viscosity of the working fluid.

The silk solution is removed from the dialysis cassettes and transferred to centrifuge tubes. The material is then centrifuged for 20 minutes at 5-10° C. (11,000 RPM) two times. After centrifugation, the silk solution concentration is determined. Typically, a solution concentration of 6-8% w/v results from the process. For some applications, higher concentrations are desired. To concentrate the solution, the standard protocol is to conduct another dialysis stage in which the silk solution is inserted into dialysis cassettes. The material is then dialyzed against PEO (PolyEthylene Oxide), which causes water to be removed from the silk solution due to osmotic pressure. Varying concentrations, typically up to 20% w/v, can be achieved by dialyzing for varying lengths of time.

To improve the through-put and efficiency, an automated water change system consists of an 8 L capacity acrylic tank with 8 cassette holders, spaced to allow exchange of water and sufficient room for cassettes to pressurize without contacting each other. The water change system greatly reduces the amount of human interaction required and enforces the proper water change volumes and intervals. A simple controller (adapted from a commercial controller used to operate automated lawn sprinklers) is used to time water changes. When a water change is initiated, the shut-off valve is opened and the water drained completely and refilled. The controller is programmed to make water changes automatically every 6 hours. To achieve sufficient dialysis, eight total tank flushes are completed. About 48 hours is required for dialysis.

Traditional dialysis cassettes are effective for removing the LiBr. The present disclosure encompasses the insight, however, that such dialysis cassettes may have a number of disadvantages, especially with respect to implementation in an automated silk solution process. For example, the present disclosure appreciates that small access ports on the corners of the cassette frame necessitate that a small-gauge needle be used to inject and remove silk solution. Injecting and/or removal silk solution from such cassettes can generate shearing effects, which is a concern for silk. With shearing forces, random coil conformation of freshly dissolved silk fibroin can be converted to a higher-order conformation, such as a crystalline beta-sheet conformation. Given the self-assembly propensity of silk solution, shearing induced within a small needle can cause at least partial conversion of random coil conformation of a solution to a more ordered conformation, which can negatively affect shelf life and the solution properties needed for various applications.

The present disclosure encompasses the insight that cassettes designed for batch processing of fixed volumes of solution create a potential for individual cassettes to fail (typically through membrane tearing at the frame edges), which leads to wasting of solution. Also, such cassettes typically require hands-on manipulation, with potentially significant force to inject and remove solution. The present disclosure appreciates that, for silk solutions, such steps may have negative impacts on the silk in the solutions, and/or on features of the solutions relevant to their performance in one or more applications.

Commercial Tangential Flow Filtration (or crossflow filtration) systems (TFF systems) are available. Typically, a solution is passed across a filter membrane at a positive pressure compared to the permeate side. Material which is smaller than the membrane pore size passes through the membrane, while the remainder stays on the supply side (called “retentate”). By flowing along (tangentially) the membrane, any trapped particles are flushed away. The key technology is the TFF tube that is held in a vertical configuration with separate inlet and outlets for the solution flows and rinse water flows. In both cases, peristaltic pumps provide flow control and ensure positive pressure on the supply side.

Silk solutions are often too viscous and sensitive to shear-induced conformation changes for the TFF systems to effectively work. Even after highly diluting the silk solution feedstock, clogging of TFF cartridges and/or particle formation in the solution due to shearing were observed. Silk solutions that did make it through the TFF systems tended to be highly dilute (−1% w/v). TFF systems could not handle the high viscosity and sensitivity to shear-induced conformation changes in silk.

Prior systems, such as commercial TFF systems, maximize efficiency through high pressure, high flow, and a high surface area to retain volume ratio. A high surface area to retain volume ratio results in extremely small channel dimensions and narrow gaps between the filtering elements.

The present disclosure encompasses a recognition that silk fibroin solutions are sensitive to shear. When silk fibroin solutions are processed in such prior systems, for example, when the structure and/or geometry of the apparatus includes small channel dimensions, when the structure and/or geometry of the apparatus includes narrow gaps between the filtering elements, where the silk fibroin solutions are processed under high pressure and/or high flow conditions, there is a tendency for formation of large aggregates of silk protein, thereby resulting in solutions with less favorable high order configurations of silk and/or rapid fouling of a filtering membrane.

In some embodiments, the present disclosure provides systems for purifying silk fibroin solutions. In some embodiments, the present disclosure provides systems for automated preparation of purified silk fibroin solutions. In some embodiments, provided silk solution purification systems dialyze a dissolved silk fibroin solution without either clogging a porous membrane or generating shear-induced conformation changes. In some embodiments, a dissolved silk fibroin solution contains salts, solvent, contaminants and/or ions does not need to be diluted to process in provided silk solution purification systems.

In some embodiments, the present disclosure provides systems for automated preparation of purified silk fibroin solutions. In some embodiments, provided silk purification systems include a dual-chamber element.

### Dual-Chamber Element

In some embodiments, provided systems for silk purification include a dual-chamber element.

In some embodiments, a dual-chamber element includes a first chamber and a second chamber.

In some embodiments, first and second chambers are defined by walls. In some embodiments, first and second chamber walls include metal, glass, plastic, natural polymers, synthetic polymers or combinations thereof.

In some embodiments, a first chamber is shaped. In some embodiments, a first chamber is circular, rectangular, triangular, or any other shape. In some embodiments, a first chamber is elongated. In some embodiments, a first chamber is hollow, for example a hollow cylinder or tube. In some embodiments, a second chamber is shaped. In some embodiments, a second chamber is circular, rectangular, triangular, or any other shape. In some embodiments, a second chamber is elongated. In some embodiments, a second chamber is hollow, for example a hollow cylinder or tube. In some embodiments, first and second chambers are substantially tubular.

In some embodiments, a dual-chamber element is defined by its length. In some embodiments, a dual-chamber element has a length within the range of about 5 cm to about 5 m. In some embodiments, a dual-chamber element has a length within a range bounded by a lower length and an upper length, the lower length being shorter than the upper length. In some embodiments, the lower length is about 5 cm, about 10 cm, about 15 cm, about 20 cm, about 25 cm, about 30 cm, about 35 cm, about 40 cm, about 45 cm, about 50 cm, about 55 cm, about 60 cm, about 65 cm, about 70 cm, about 75 cm, about 80 cm, about 85 cm, about 90 cm, about 1 m, about 1.2 m, about 1.3 m, about 1.4 m, about 1.5 m, about 1.6 m, about 1.7 m, about 1.8 m, about 1.9 m, about 2.1 m, about 2.2 m, about 2.3 m, about 2.4 m, about 2.5 m, about 2.6 m, about 2.7 m, about 2.8 m, about 2.9 m, about 3.0 m, about 3.1 m, about 3.2 m, about 3.3 m, about 3.4 m, about 3.5 m, about 3.6 m, about 3.7 m, about 3.8 m, about 3.9 m, about 4.0 m, about 4.1 m, about 4.2 m, about 4.3 m, about 4.4 m, about 4.5 m, about 4.6 m, about 4.7 m, about 4.8 m, about 4.9 m, or about 5.0 m. In some embodiments, the upper length is about 5 m, about 4.9 m, about 4.8 m, about 4.7 m, about 4.6 m, about 4.5 m, about 4.4 m, about 4.3 m, about 4.2 m, about 4.1 m, about 4 m, about 3.9 m, about 3.8 m, about 3.7 m, about 3.6 m, about 3.5 m, about 3.4 m, about 3.3 m, about 3.2 m, about 3.1 m, about 3 m, about 2.9 m, about 2.8 m, about 2.7 m, about 2.6 m, about 2.5 m, about 2.4 m, about 2.3 m, about 2.2 m, about 2.1 m, about 2 m, about 1.9 m, about 1.8 m, about 1.7 m, about 1.6 m, about 1.5 m, about 1.4 m, about 1.3 m, about 1.2 m, about 1.1 m, about 1 m, about 95 cm, 90 cm, about 85 cm, about 80 cm, about 75 cm, about 70 cm, about 65 cm, about 60 cm, about 55 cm, about 50 cm, about 45 cm, about 40 cm, about 35 cm, about 30 cm, about 25 cm, about 20 cm, about 15 cm, about 10 cm, or about 5 cm.

In some embodiments, a length of a dual-chamber element is about 5 m, about 4 m, about 3 m, about 2 m, about 1 m, about 95 cm, 90 cm, about 85 cm, about 80 cm, about 75 cm, about 70 cm, about 65 cm, about 60 cm, about 55 cm, about 50 cm, about 45 cm, about 40 cm, about 35 cm, about 30 cm, about 25 cm, about 20 cm, about 15 cm, about 10 cm, or about 5 cm. In some embodiments, a first chamber is the same length or about the same length as a second chamber. In some embodiments, first and second chambers are different lengths.

In some embodiments, first and second chambers are defined by width and/or depth. In some embodiments, a depth is about 1 m, about 95 cm, 90 cm, about 85 cm, about 80 cm, about 75 cm, about 70 cm, about 65 cm, about 60 cm, about 55 cm, about 50 cm, about 45 cm, about 40 cm, about 35 cm, about 30 cm, about 25 cm, about 20 cm, about 15 cm, about 10 cm, or about 5 cm. In some embodiments, a width is about 1 m, about 95 cm, 90 cm, about 85 cm, about 80 cm, about 75 cm, about 70 cm, about 65 cm, about 60 cm, about 55 cm, about 50 cm, about 45 cm, about 40 cm, about 35 cm, about 30 cm, about 25 cm, about 20 cm, about 15 cm, about 10 cm, or about 5 cm.

In some embodiments, a dual-chamber element has a width and/or depth within a range bounded by a lower width and/or depth and an upper width and/or depth, the lower width and/or depth being smaller than the upper width and/or depth. In some embodiments, the lower width and/or depth is about 5 cm, about 10 cm, about 15 cm, about 20 cm, about 25 cm, about 30 cm, about 35 cm, about 40 cm, about 45 cm, about 50 cm, about 55 cm, about 60 cm, about 65 cm, about 70 cm, about 75 cm, about 80 cm, about 85 cm, about 90 cm, about 1 m, about 1.2 m, about 1.3 m, about 1.4 m, about 1.5 m, about 1.6 m, about 1.7 m, about 1.8 m, about 1.9 m, about 2.1 m, about 2.2 m, about 2.3 m, about 2.4 m, about 2.5 m, about 2.6 m, about 2.7 m, about 2.8 m, about 2.9 m, about 3.0 m, about 3.1 m, about 3.2 m, about 3.3 m, about 3.4 m, about 3.5 m, about 3.6 m, about 3.7 m, about 3.8 m, about 3.9 m, about 4.0 m, about 4.1 m, about 4.2 m, about 4.3 m, about 4.4 m, about 4.5 m, about 4.6 m, about 4.7 m, about 4.8 m, about 4.9 m, or about 5.0 m. In some embodiments, the upper width and/or depth is about 5 m, about 4.9 m, about 4.8 m, about 4.7 m, about 4.6 m, about 4.5 m, about 4.4 m, about 4.3 m, about 4.2 m, about 4.1 m, about 4 m, about 3.9 m, about 3.8 m, about 3.7 m, about 3.6 m, about 3.5 m, about 3.4 m, about 3.3 m, about 3.2 m, about 3.1 m, about 3 m, about 2.9 m, about 2.8 m, about 2.7 m, about 2.6 m, about 2.5 m, about 2.4 m, about 2.3 m, about 2.2 m, about 2.1 m, about 2 m, about 1.9 m, about 1.8 m, about 1.7 m, about 1.6 m, about 1.5 m, about 1.4 m, about 1.3 m, about 1.2 m, about 1.1 m, about 1 m, about 95 cm, 90 cm, about 85 cm, about 80 cm, about 75 cm, about 70 cm, about 65 cm, about 60 cm, about 55 cm, about 50 cm, about 45 cm, about 40 cm, about 35 cm, about 30 cm, about 25 cm, about 20 cm, about 15 cm, about 10 cm, or about 5 cm.

In some embodiments, a first and/or a second chamber is circular or tubular. In some embodiments, a circular or tubular chamber is defined by a diameter. In some embodiments, a diameter is about 1 m, about 95 cm, 90 cm, about 85 cm, about 80 cm, about 75 cm, about 70 cm, about 65 cm, about 60 cm, about 55 cm, about 50 cm, about 45 cm, about 40 cm, about 35 cm, about 30 cm, about 25 cm, about 20 cm, about 15 cm, about 10 cm, or about 5 cm.

In some embodiments, a width of first and second chambers is the same. In some embodiments, a width of first and second chambers is the different. In some embodiments, a depth of first and second chambers is the same. In some embodiments, a depth of first and second chambers is the different.

In some embodiments, a first chamber has ends. In some embodiments, a first chamber has first and second ends. In some embodiments, a first chamber is open at its ends. In some embodiments, an end closes or seal a first chamber. In some embodiments, an end is or includes, for example, a plastic, rubber, Teflon, or natural or synthetic polymer. In some embodiments, a seal at an end forms by compression. In some embodiments, a seal at an end forms by capping. In some embodiments, a seal at an end forms by threading an end on a first chamber. In some embodiments, a seal is a removable seal. In some embodiments, a first chamber is selectively open at its ends. In some embodiments, ends include ports having a valve for control.

In some embodiments, a first chamber is enclosed within or by a second chamber. In some embodiments, when a first chamber is enclosed within or by a second chamber, an outer surface of a first chamber is a common surface or wall between a first chamber and a second chamber. In some embodiments, a tubular porous membrane is surrounded by a rigid outer tube to create separate chambers.

In some embodiments, at least one common surface or wall between a first chamber and a second chamber is porous. In some embodiments, first and second chambers are separated by a porous membrane.

In some embodiments, systems for automated preparation of purified silk fibroin solutions as provided herein are characterized in that they retain silk proteins in a molecular weight range between about 1 kDa and about 400 kDa. In some embodiments, a porous membrane is or includes a permeable membrane, a semi-permeable membrane, a selectively permeable membrane, a dialysis membrane, cellulose tubing, regenerated cellulose tubing, or SnakeSkin tubing. In some embodiments, a porous membrane includes pores. In some embodiments, pores are defined by size. In some embodiments, pores are sized to retain proteins. In some embodiments, pores are sized to retain proteins above about 1 kDa. In some embodiments, pores are sized to retain proteins between about 1 kDa and about 400 kDa. In some embodiments, pores are sized to retain proteins between about 1 kDa and about 100 kDa.

In some embodiments, systems for automated preparation of purified silk fibroin solutions as provided herein are characterized it that when a dissolved silk fibroin solution having a viscosity between about 1.0 cP and 30 cP flows into or through a first chamber, salts, contaminants, solvents, and/or ions cross a porous membrane into a second chamber, and silk proteins from are retained in a retentate solution in a first chamber. In some embodiments, systems for automated preparation of purified silk fibroin solutions as provided herein are characterized it that when a dissolved silk fibroin solution having a viscosity between about 2.0 cP and 20 cP flows into or through a first chamber, salts, contaminants, solvents, and/or ions cross a porous membrane into a second chamber, and silk proteins from are retained in a retentate solution in a first chamber. In some embodiments, systems for automated preparation of purified silk fibroin solutions as provided herein are characterized it that when a dissolved silk fibroin solution having a viscosity between about 3.0 cP and 8.0 cP flows into or through a first chamber, salts, contaminants, solvents, and/or ions cross a porous membrane into a second chamber, and silk proteins from are retained in a retentate solution in a first chamber.

In some embodiments a silk solution is a dissolved silk fibroin solution. In some embodiments, a silk solution is a dissolved silk fibroin solution as is above described in more detail. In some embodiments, a silk solution is a silk fibroin solution that is partially or mostly purified. In some embodiments, a dissolved silk fibroin solution includes salts, contaminants, solvents, and/or ions. In some embodiments, a first chamber and a second chamber are adjacent to one another. In some embodiments, first and second chambers are separated by a common surface or wall. In some embodiments, first and second chambers share at least one surface or wall in common.

In some embodiments a dissolved silk fibroin solution is stored in a reservoir. In some embodiments, a dissolved silk fibroin solution flows from a first reservoir fills a first chamber when it is introduced, enters, or flows through an opening at an end of a first chamber. In some embodiments, a dissolved silk fibroin solution is introduced, enters, or flows through an opening at a first end of a first chamber and flows through a first chamber and out an opening at a second end.

In some embodiments, movement of particles and materials within a dissolved silk fibroin solution operates on diffusion such that molecules random move from an area of higher concentration to an area of lower concentration. In some embodiments, movement of a solvent (e.g. water) within a dissolved silk fibroin solution operates on osmosis such that a solvent moves across a porous membrane from an area of weaker concentration (hypotonic) to an area of stronger concentration (hypertonic). Osmotic pressure is a pressure required to maintain an equilibrium, with no net movement of solvent. Osmotic pressure is a colligative property, meaning that solutions depend on a ratio of a number of solute particles to a number of solvent molecules in a solution, molar concentration, and not on a type or identity of chemical species present. In some embodiments, movement of particles and materials within a dissolved silk fibroin solution operates on dialysis such that particle and materials within a dissolved silk fibroin solution, for example, including salts, contaminants, solvents, and/or ions diffuse across a porous membrane. In some embodiments, particles and materials move across a porous membrane from an area of weaker concentration to an area of stronger. In some embodiments, a dissolved silk fibroin solution flows in a first chamber.

In some embodiments, a dissolved silk fibroin solution flows in a first chamber along a porous membrane separating a first chamber from a second chamber. In some embodiments, a dissolved silk fibroin solution exerts pressure on a porous membrane. In some embodiments, a dissolved silk fibroin solution exerts pressure on a porous membrane in a direction that is normal relative to a porous membrane. In some embodiments, a dissolved silk fibroin solution flows in a direction that is tangential relative filtration.

In some embodiments, a dissolved silk fibroin solution flows in a first chamber along a porous membrane separating a first chamber from a second chamber. In some embodiments, when material in such a solution is smaller than a membrane pore size, material passes into such a porous membrane. In some embodiments, when material in such a solution is smaller than a membrane pore size, material passes through such a porous membrane and into a second chamber.

In some embodiments, when a dissolved silk fibroin solution flows through a first chamber it contacts a surface including a porous membrane. In some embodiments, material in a dissolved silk fibroin solution is smaller than a membrane pore size. In some embodiments, when a dissolved silk fibroin solution flows along a porous membrane material in a dissolved silk fibroin solution that is smaller than porous membranes pores, such material passes into it. In some embodiments, when a dissolved silk fibroin solution flows along a porous membrane material in a dissolved silk fibroin solution passes through it. In some embodiments, material in a dissolved silk fibroin solution that passes through includes salts, contaminants, solvents, and/or ions. In some embodiments, material in a dissolved silk fibroin solution that is retained is in a first chamber includes silk proteins that are about as large and larger than a membrane pore size.

In some embodiments, a second chamber has ends. In some embodiments, a second chamber has first and second ends. In some embodiments, a second chamber is open at its ends. In some embodiments, an end closes or seal a second chamber. In some embodiments, an end is or includes, for example, a plastic, rubber, Teflon, or natural or synthetic polymer. In some embodiments, a seal at an end forms by compression. In some embodiments, a seal at an end forms by capping. In some embodiments, a seal at an end forms by threading an end on a second chamber. In some embodiments, a seal is a removable seal. In some embodiments, a second chamber is selectively open at its ends. In some embodiments, ends include ports having a valve for control. In some embodiments, a second chamber is selectively open at its ends. In some embodiments, ends include ports having a valve for control.

In some embodiments, a second chamber includes a solution. In some embodiments, a second chamber does not contain a solution. In some embodiments, a second chamber solution is or includes a dialysate. In some embodiments, a dialysate solution is or includes water, polyethylene oxide, glycerol, polyvinyl alcohol, or hygroscopic polymer fluids. In some embodiments, a second chamber contains a gas, such as air.

In some embodiments, a solution in a second chamber acts as a dialysate. In some embodiments, a dialysate solution acts to draw material, including for example salts, contaminants, solvents, and/or ions from a dissolved silk fibroin solution. In some embodiments, a dialysate solution acts to draw material, including for example salts, contaminants, solvents, and/or ions from a porous membrane separating first and second chambers.

In some embodiments, a dialysate solution is a counter-flow fluid that flows in the second chamber in a direction opposite to that of a dissolved silk fibroin solution flowing in a first chamber. In some embodiments, when a counter-flow fluid that flows in a second chamber in a direction opposite to that of a dissolved silk fibroin solution flowing in a first chamber, salts, contaminants, solvents, and/or ions are drawn out of such a solution through a porous membrane and removed when the counter-flow fluid is extracted from the second chamber. In some embodiments, a dissolved silk fibroin solution flows through a first chamber with a positive pressure. In some embodiments, a dissolved silk fibroin solution flows through a first chamber with a positive pressure relative to a pressure of a second chamber. In some embodiments, a transmembrane pressure is an average pressure differential between a first chamber and a second chamber. In some embodiments, a transmembrane pressure is a force that pushes salts, contaminants, solvents, and/or ions from a first chamber through a porous membrane to a second chamber. In some embodiments, a transmembrane pressure is between about 0.10 psi-about 50 psi.

In some embodiments, a dissolved silk fibroin solution flows at a flow rate. In some embodiments, a flow rate is a rate at which a dissolved silk fibroin solution flows along the membrane surface. In some embodiments, a flow rate is between about 1 mL/hr and about 1000 mL/hr. In some embodiments, a flow rate is between about 10 cm³/sec and about 100 cm³/sec. In some embodiments, a transmembrane pressure and/or flow is controlled via feed pump, such as for example a peristaltic pump.

In some embodiments, a dissolved silk fibroin solution is at room temperature. In some embodiments, a dissolved silk fibroin solution is at a temperature between about 15° C. and about 50° C.

In some embodiments, viscosity, flow, pressure, temperature will vary with geometry of systems for automated preparation of purified silk fibroin solutions. In some embodiments, geometric dimensions includes length, width, and depth of first and second chambers.

In some embodiments, geometry includes a gap between a porous membrane and an outside wall of a second chamber. In some embodiments, a gap is defined between a porous membrane separating an inner wall of a second chamber and an outer wall of a porous membrane. In some embodiments a gap between a membrane and an outer wall has a size within a range of about less than about 1 mm to about 20 mm. In some embodiments, such a gap has a size within a range bounded by a lower length and an upper length, the lower length being smaller than the upper length. In some embodiments, a lower length of a gap is less than about 1 mm, about 1.5 mm, about 2 mm, about 2.5 mm, about 3 mm, about 3.5 mm, about 4 mm, about 4.5 mm, about 5 mm, about 5.5 mm, about 6 mm, about 6.5 mm, about 7 mm, about 7.5 mm, about 8 mm, about 8.5 mm, about 9 mm, about 9.5 mm, about 10 mm, about 10.5 mm, about 11 mm, about 11.5 mm, about 12 mm, about 12.5 mm, about 13 mm, about 13.5 mm, about 14 mm, about 14.5 mm, about 15 mm, about 15.5 mm, about 16 mm, about 16.5 mm, about 17 mm, about 17.5 mm, about 18 mm, about 18.5 mm, about 19 mm, about 19.5 mm, or about 20 mm. In some embodiments, the upper length of a gap is about 20 mm, about 19.5 mm, about 19 mm, about 18.5 mm, about 18 mm, about 17.5 mm, about 17 mm, about 16.5 mm, about 16 mm, about 15.5 mm, about 15 mm, about 14.5 mm, about 14 mm, about 13.5 mm, about 13 mm, about 12.5 mm, about 12 mm, about 11.5 mm, about 11 mm, about 10.5 mm, about 10 mm, about 9.5 mm, about 9 mm, about 8.5 mm, about 8 mm, about 7.5 mm, about 7 mm, about 6.5 mm, about 6 mm, about 5.5 mm, about 5 mm, about 4.5 mm, about 4 mm, about 3.5 mm, about 3 mm, about 2.5 mm, about 2 mm, about 1.5 mm, or less than about 1 mm.

In some embodiments, a gap between a membrane and an outer wall has a size that is at least about 0.1 mm, at least about 0.2 mm, at least about 0.3 mm, at least about 0.4 mm, at least about 0.5 mm, at least about 0.6 mm, at least about 0.7 mm, at least about 0.8 mm, at least about 0.9 mm, at least about 1.0 mm, at least about 1.1 mm, at least about 1.2 mm, at least about 1.3 mm, at least about 1.4 mm, at least about 1.5 mm, at least about 1.6 mm, at least about 1.7 mm, at least about 1.8 mm, at least about 1.9 mm, at least about 2.0 mm, at least about 2.5 mm, at least about 3.0 mm, at least about 3.5 mm, at least about 4.0 mm, at least about 4.5 mm, at least about 5.0 mm, at least about 5.5 mm, at least about 6.0 mm, at least about 6.5 mm, at least about 7.0 mm, at least about 7.5 mm, at least about 8.0 mm, at least about 8.5 mm, at least about 9.0 mm, at least about 9.5 mm, at least about 10.0 mm, at least about 11.0 mm, at least about 12.0 mm, at least about 13.0 mm, at least about 14.0 mm, at least about 15.0 mm, at least about 16.0 mm, at least about 17.0 mm, at least about 18.0 mm, at least about 19.0 mm, or at least about 20.0 mm.

In some embodiments, a silk fibroin solution formation will vary with differing gap distance, flow rate, pressure, dissolved silk fibroin solution concentration, and salt concentration.

In some embodiments, lowering the flow and pressure reduces formation of less favorable solutions containing higher order configurations of silk and/or clogged membranes. In some embodiments, a gap reduces flow and pressure. In some embodiments, a gap reduces shear sensitivity in a dissolved silk fibroin solution. In some embodiments, a gap reduces a tendency of a dissolved silk fibroin solution to form large aggregates.

In some embodiments, geometry includes a ratio of surface area to retained volume. In some embodiments a ratio is in a range of about 0.1:1 to about 20:1 In some embodiments a ratio is about 0.1:1, about 0.2:1, about 0.3:1, about 0.4:1, about 0.5:1, about 0.6:1, about 0.7:1, about 0.8:1, about 0.9:1, about 1:1, about 1.1:1, about 1.2:1, about 1.3:1, about 1.4:1, about 1.5:1, about 1.6:1, about 1.7:1, about 1.8:1, about 1.9:1, about 2:1, about 2.5:1, about 3:1, about 3.5:1, about 4:1, about 4.5:1, about 5:1, about 5.5:1, about 6:1, about 6.5:1, about 7:1, about 7.5:1, about 8:1, about 8.5:1, about 9:1, about 9.5:1, about 10:1, about 10.5:1, about 11:1, about 11.5:1, about 12:1, about 12.5:1, about 13:1, about 13.5:1, about 14:1, about 14.5:1, about 15:1, about 15.5:1, about 16:1, about 16.5:1, about 17:1, about 17.5:1, about 18:1, about 18.5:1, about 19:1, about 19.5:1, or about 20:1.

In some embodiments, the present disclosure provides systems including a smaller ratio of surface area to retained volume. In some embodiments, a smaller ratio results in a gap between a porous membrane and an outer wall.

In some embodiments, a vacuum pump removes air pockets. In some embodiments, air pockets can cause a buildup of pressure. In some embodiments, increased pressure may induce shear. In some embodiments, removing air pockets reduces pressure buildup thereby reducing the likelihood of shear.

In some embodiments, salts, contaminants, solvents, and/or ions may collect near the bottom of a second chamber. In some embodiments, such collecting reduces a porous membrane's efficiency. In some embodiments, tilting a dual-chamber element reduces salts, contaminants, solvents, and/or ions collecting. In some embodiments, a dual-chamber element is tilted away from normal. In some embodiments, a dual-chamber element is tilted away from normal at an angle within a range of about 10° to about 80°. In some embodiments, a dual-chamber element is tilted away from normal at an angle of about 10°, about 20°, about 30°, about 35°, about 40°, about 45°, about 50°, about 55°, about 60°, about 70°, or about 80°.

In some embodiments, provided systems include at least one dual-chamber element. In some embodiments, a silk solution purification system includes at least two dual-chamber elements. In some embodiments, provided systems include multiple dual-chamber elements.

In some embodiments, a silk solution purification system includes a combination of multiple dual-chamber elements. In some embodiments, multiple dual-chamber elements operate in parallel. In some embodiments, multiple dual-chamber elements operate in series. In some embodiments, when multiple dual-chamber elements are in series, each dual-chamber element works to further purify a silk fibroin solution thereby reducing the concentration salts, contaminants, solvents, and/or ions therein. In some embodiments, when multiple dual-chamber elements are used in series, silk fibroin solutions are purified to levels of salts, contaminants, solvents, and/or ions that are unexpectedly low and not seen before in the art. In some embodiments, when multiple dual-chamber elements are used in series, silk fibroin solutions are purified to levels of salts, contaminants, solvents, and/or ions that are unexpectedly low and previously not seen in the art without effects of protein aggregation due to shear.

In some embodiments, when provided automated silk purification systems include at least two or more dual-chamber elements, a mixing stage or reservoir is arranged at an output of a first dual-chamber element. In some embodiments, when provided automated silk purification systems include two or more dual-chamber elements, a mixing stage or reservoir is arranged at an input of any dual-chamber element.

In some embodiments, systems with at least two dual-chamber elements include dual-chamber elements that are each about a same length. In some embodiments, systems with at least two dual-chamber elements include dual-chamber elements that are each different in length.

In some embodiments, provided silk solution purification systems produce silk solutions including a population of silk fragments. In some embodiments, produced or retained silk fragments are part of a resultant solution. In some embodiments, resultant solutions may differ according to a molecular weight of their silk fragments. In some embodiments, resultant silk solutions include a uniform distribution of silk fibroin fragments or a non-uniform distribution of silk fibroin fragments.

In some embodiments, technologies and methods of forming silk solutions that differ according to a molecular weight of its silk fragments, include for example, controlling fragment size through boiling. In some embodiments, as provided herein, boiling time (mb) at least partially defines silk fragment size, molecular weight, and/or a range of molecular weight fragments of silk.

In some embodiments, systems for automated preparation of purified silk fibroin solutions produce silk fibroin solutions that are characterized in that they include a non-uniform collection of silk fragments having molecular weights in a range of about 1 kDa to about 400 kDa. In some embodiments, silk solutions formed by methods and technologies as described herein include silk fibroin fragments having a non-uniform collection of molecular weights. In some embodiments, silk solutions including silk fragments with a non-uniform distribution of molecular weights are polydisperse silk solutions.

In some embodiments, systems for automated preparation of purified silk fibroin solutions produce silk fibroin solutions that are characterized in that they include a uniform collection of silk fragments having molecular weights in a range of about 1 kDa to about 400 kDa. In some embodiments, a silk solution formed by methods and technologies as described herein includes silk fibroin fragments having a particular molecular weight or have a narrow range of molecular weights. In some embodiments, a silk solution including silk fragments with a particular molecular weight or having a narrow range of molecular weights are monodisperse silk solutions.

In some embodiments, systems for automated preparation of purified silk fibroin solutions produce uniform, or monodisperse silk solutions. In some embodiments, resultant silk solutions are discretely monodisperse around a single molecular weight value. For example, in some embodiments, systems for automated preparation of purified silk fibroin solutions, produce silk fibroin solutions that include a uniform collection of silk fibroin fragments having a molecular weights centered around a single molecular weight in a range of about 1 kDa to about 400 kDa. In some embodiments, a single molecular weight is an average, mean, mode, median molecular weight. In some embodiments, an average single molecular weight includes a single standard deviation, two standard deviations, three standard deviations, or four standard deviations. In some embodiments, a single molecular weight is an average, for example, about 1 kDa, about 2 kDa, about 3 kDa, about 4 kDa, about 5 kDa, about 6 kDa, about 7 kDa, about 8 kDa, about 9 kDa, about 10 kDa, about 11 kDa, about 12 kDa, about 13 kDa, about 14 kDa, about 15 kDa, about 16 kDa, about 17 kDa, about 18 kDa, about 19 kDa, about 20 kDa, about 21 kDa, about 22 kDa, about 23 kDa, about 24 kDa, about 25 kDa, about 30 kDa, about 35 kDa, about 40 kDa, about 45 kDa, about 50 kDa, about 55 kDa, about 60 kDa, about 65 kDa, about 70 kDa, about 75 kDa, about 80 kDa, about 85 kDa, about 90 kDa, about 95 kDa, about 100 kDa, about 105 kDa, about 110 kDa, about 115 kDa, about 120 kDa, about 125 kDa, about 130 kDa, about 135 kDa, about 140 kDa, about 145 kDa, about 150 kDa, about 155 kDa, about 160 kDa, about 165 kDa, about 170 kDa, about 175 kDa, about 180 kDa, about 185 kDa, about 190 kDa, about 195 kDa, about 200 kDa, about 225 kDa, about 250 kDa, about 275 kDa, about 300 kDa, about 325 kDa, about 350 kDa, about 375 kDa, or about 400 kDa.

In some embodiments, systems for automated preparation of purified silk fibroin solutions produce uniform, or monodisperse silk solutions. In some embodiments, silk solutions formed are continuously monodisperse within a range of molecular weights. In some embodiments, systems for automated preparation of purified silk fibroin solutions form uniform, or monodisperse silk solutions, for example, of about 2 kDa to about 50 kDa, 2 kDa to about 100 kDa, about 2 kDa to about 125 kDa, about 2 kDa to about 150 kDa, about 2 kDa to about 175 kDa, about 2 kDa to about 200 kDa, about 2 kDa to about 250 kDa, about 50 kDa to about 100 kDa, about 50 kDa to about 150 kDa, about 50 kDa to about 200 kDa, about 50 kDa to about 250 kDa, about 50 kDa to about 300 kDa, about 100 kDa to about 150 kDa, about 100 kDa to about 200 kDa, about 100 kDa to about 250 kDa, about 100 kDa to about 300 kDa, about 100 kDa to about 350 kDa, about 150 kDa to about 200 kDa, about 150 kDa to about 250 kDa, about 150 kDa to about 300 kDa, about 150 kDa to about 350 kDa, about 150 kDa to about 400 kDa, about 200 kDa to about 250 kDa, about 200 kDa to about 300 kDa, about 200 kDa to about 350 kDa, about 200 kDa to about 400 kDa, about 250 kDa to about 300 kDa, about 250 kDa to about 350 kDa, about 250 kDa to about 400 kDa, about 300 kDa to about 350 kDa, about 300 kDa to about 400 kDa, or about 350 to about 400 kDa.

In some embodiments, systems for automated preparation of purified silk fibroin solutions produce silk solutions that are characterized by a polydispersity index. A polydispersity index represents a distribution by molecular mass of silk fibroin fragments.

In some embodiments, size distribution of silk fragments may be characterized by a polydispersity index (PDI). In some embodiments, PDI of silk fragments is determined by methods commonly known by one of ordinary skill in the art, for example, by dynamic light scattering (DLS) measurement. With regard to DLS used for particle size determinations, the common use of second or third order cumulant analysis to fit the autocorrelation function leads to the values of PDI.

In some embodiments, an absolute value of PDI determined from this method is in a range from zero and higher. In some embodiments, small values indicate narrower distributions. For example, PDI in a range of about 0 to about 0.3 or from about 0 to about 0.4 presents relatively monodisperse particle size distributions. In some embodiments, a non-uniform collection of molecular weights results in higher polydispersity index. This criterion has been generally accepted in the art of dynamic light scattering for particle size determinations.

In some embodiments, silk solution purification systems include cameras, chemical analysis equipment, sensors, and/or techniques to measure silk solution properties, for example, silk concentration, salt concentration, ion concentration, a concentration of higher order configurations of silk, and/or turbidity. In some embodiments, silk solution purification systems include cameras, chemical analysis equipment, sensors, and/or techniques to measure silk solution properties in situ. Such monitoring and analysis equipment is known in the art.

In some embodiments, a purified silk fibroin solution is stored in a reservoir. In some embodiments, a stored purified silk fibroin solution is fed to a concentrating system. In some embodiments, an automated silk purification system is integrated with a silk fibroin solution concentrating system.

### Methods of Purifying a Silk Fibroin Solution

In some embodiments, the present disclosure provides methods for automated preparation of purified silk fibroin solutions.

In some embodiments, methods include providing a dissolved silk fibroin solution for purification. In some embodiments, a dissolved silk fibroin solution includes salts, solvents, contaminants, and/or ions.

In some embodiments, methods include providing an automated silk purification system. In some embodiments, methods include providing at least one dual-chamber element.

In some embodiments, methods include introducing or flowing a dissolved silk fibroin solution into a first chamber of a dual-chamber element of an automated silk purification system. In some embodiments, methods include flowing a dissolved silk fibroin solution through a first chamber of a dual-chamber element of an automated silk purification system. In some embodiments, methods include pumping a dissolved silk fibroin solution into a first chamber of an automated silk purification system.

In some embodiments, a flowing dissolved silk fibroin solution is characterized by a pressure and a flow rate. In some embodiments, a pressure and/or flow rate of a dissolved silk fibroin solution is below a threshold that induces silk protein aggregation.

In some embodiments, methods include contacting a dissolved silk fibroin solution with a porous membrane. In some embodiments, methods include flowing a dissolved silk fibroin solution over a porous membrane.

In some embodiments, methods include providing a fluid in a second chamber of an automated silk purification system. In some embodiments, a fluid is water. In some embodiments, methods include introducing or flowing a fluid into a second chamber of an automated silk purification system. In some embodiments, methods include flowing a fluid through a second chamber of an automated silk purification system. In some embodiments, a fluid is a counter-flow fluid. In some embodiments, a counter-flow fluid flows in a second chamber in a direction that opposes a flow of a dissolved silk fibroin solution in a first chamber.

In some embodiments, methods include retaining silk proteins in a dissolved silk fibroin solution in or flowing through a first chamber. In some embodiments, methods include extracting a fluid from a second chamber including salts, solvent, contaminants, and/or ions that entered or crossed a porous membrane separating first and second chambers.

In some embodiments, methods include tilting a dual-chamber element away from normal.

In some embodiments, methods include providing an automated silk purification system including at least two dual-chamber elements. In some embodiments, methods include providing an automated silk purification system including at least two dual-chamber elements where each dual-chamber element is a same length. In some embodiments, methods include providing an automated silk purification system including at least two dual-chamber elements where each dual-chamber element is a different length. In some embodiments, methods include providing an automated silk purification system including at least two dual-chamber elements where at least one dual-chamber element is a different length.

In some embodiments, methods include connecting dual-chamber elements. In some embodiments, methods include connecting dual-chamber elements in parallel. In some embodiments, methods include connecting dual-chamber elements in series.

In some embodiments, methods include pumping a dissolved silk fibroin solution into a first chamber of each of at least two dual-chamber elements. In some embodiments, methods include pumping a dissolved silk fibroin solution into a first chamber of at least one dual-chamber elements of at least two dual-chamber elements.

In some embodiments, methods include tilting each dual-chamber element away from normal.

In some embodiments, methods include detecting, in situ detecting and/or monitoring a silk solution concentration, a salt concentration, an ion concentration, a concentration of higher order configurations of silk, and/or a silk solution turbidity. In some embodiments, methods include in situ detecting a silk solution concentration, a salt concentration, an ion concentration, a concentration of higher order configurations of silk, and/or a silk solution turbidity.

In some embodiments, methods include detecting, in situ detecting and/or monitoring using cameras, chemical analysis equipment, and/or sensors.

In some embodiments, methods include analyzing and/or a silk solution concentration, a salt concentration, an ion concentration, a concentration of higher order configurations of silk, and/or a silk solution turbidity.

In some embodiments, methods include connecting dual chamber elements, where the elements are configured to retain single molecular weight silk fragments. In some embodiments, methods include connecting dual chamber elements, where the elements are configured to retain at least two different molecular weight silk fragments. In some embodiments, methods include connecting dual chamber elements, where the elements are configured to retain more than two different molecular weight silk fragments. In some embodiments, methods and technologies provided herein tailor polydispersity of silk fibroin solutions.

### Silk Fibroin Solution Concentrating System

In some embodiments, higher concentrations are desirable for silk solution application, such as for example many silk materials and silk structural formats. In prior systems, the standard protocol for preparing higher concentration is to perform another dialysis stage. The dialyzed silk fibroin solution is introduced into dialysis cassettes. The purified silk fibroin solution is then dialyzed against PolyEthylene Oxide (PEO). PEO causes water to be removed from the purified silk fibroin solution due to osmostic pressure. Varying concentrations, typically up to 20% w/v, can be achieved by dialyzing for varying lengths of time against PEO. Of course, additional dialysis steps require additional processing and exposure to handling that increases shear induced aggregation.

In some embodiments, the present disclosure provides systems for concentrating purified silk fibroin solutions. In some embodiments, the present disclosure provides systems for automated preparation of concentrated purified silk fibroin solutions. In some embodiments, provided silk solution concentrating systems concentrate purified silk fibroin solutions without either clogging a porous membrane or generating shear-induced conformation changes.

In some embodiments, the present disclosure provides systems for automated concentrating of purified silk fibroin solutions. In some embodiments, provided silk concentrating systems include a dual-chamber element.

### Dual-Chamber Element

In some embodiments, a silk solution concentrating system includes a dual-chamber element. In some embodiments, a dual-chamber column is used to concentrate a purified silk fibroin solution. In some embodiments, a dual-chamber element includes a first chamber and a second chamber separated by a porous membrane.

In some embodiments, a silk solution concentrating system further includes a purified silk fibroin solution reservoir. In some embodiments, a purified silk fibroin solution reservoir includes a purified silk fibroin solution. In some embodiments, a purified silk fibroin solution is post-dialysis.

In some embodiments, a purified silk fibroin solution reservoir is integrated with and/or connected between a silk solution purification system and a silk solution concentrating system. In some embodiments, a purified silk fibroin solution that is output from a silk solution purification system is an input to a silk solution concentrating system.

In some embodiments, a purified silk fibroin solution has a starting concentration between less than about 1% w/v and about 10% w/v. In some embodiments, a purified silk fibroin solution has a starting concentration of between about 4% w/v and 4.5% w/v. In some embodiments, a silk solution concentrating system further includes a purified silk fibroin solution reservoir.

In some embodiments, a purified silk fibroin solution is fed into a first chamber from a top of a dual-chamber element. In some embodiments, a purified silk fibroin solution is gravity fed into a first chamber from a top of a dual-chamber element.

In some embodiments, a second chamber includes or is filled with air or a gas. In some embodiments, a second chamber includes no water or other fluids. In some embodiments, a solvent, such as for example water present in a purified silk fibroin solution that is contained in a first chamber crosses a porous membrane into a second chamber. In some embodiments, a purified silk fibroin solution is retained in a first chamber. In some embodiments, a concentrated purified silk fibroin solution is retained in a first chamber.

In some embodiments, a vertical arrangement and gravity-fed design of a silk solution concentrating system automatically separates a concentrated purified silk fibroin solution.

In some embodiments, a dual-chamber element is vertical. In some embodiments, a silk solution concentrating system includes a valve at an opening for introducing a purified silk fibroin solution. In some embodiments, a silk solution concentrating system includes at least one valve at or near a bottom of a dual-chamber element for removing a concentrated purified silk fibroin solution. In some embodiments, a silk solution concentrating system includes multiple valves at or near a bottom of a dual-chamber element and/or along a side of a dual-chamber element for removing different concentrations of a concentrated purified silk fibroin solution.

In some embodiments, a vertical arrangement and gravity-fed design of a silk solution concentrating system reduces shear relative to prior designs. In some embodiments, different concentrations of silk are extracted based on the height where a sample is present in a column by extracting through a valve at such a location.

In some embodiments, a silk solution concentrating systems include sensors or chemical analysis equipment and techniques to measure silk solution properties, for example, silk concentration, salt concentration, ion concentration, a concentration of higher order configurations of silk, and/or turbidity.

### Methods of Concentrating a Silk Fibroin Solutions

In some embodiments, the present disclosure provides methods for automated concentrating of purified silk fibroin solutions.

In some embodiments, methods include providing a silk solution for concentrating. In some embodiments, a silk solution is a purified silk fibroin solution.

In some embodiments, methods include providing a silk solution concentrating system. In some embodiments, methods include providing a dual-chamber element.

In some embodiments, methods include introducing a purified silk fibroin solution into a first chamber of a dual-chamber element of a silk solution concentrating system.

In some embodiments, methods include gravity feeding a purified silk fibroin solution into a first chamber of a dual-chamber element of a silk solution concentrating system.

In some embodiments, methods include contacting a purified silk fibroin solution with a porous membrane.

In some embodiments, methods include providing a fluid in a second chamber of a silk solution concentrating system. In some embodiments, a fluid is a gas. In some embodiments, a gas is air.

In some embodiments, methods include extracting a fluid from a second chamber including a solvent that entered or crossed a porous membrane separating first and second chambers. In some embodiments, methods include retaining silk proteins in a purified silk fibroin solution in a first chamber.

In some embodiments, methods include detecting, in situ detecting and/or monitoring a silk solution concentration. In some embodiments, methods include detecting, in situ detecting and/or monitoring using cameras, chemical analysis equipment, and/or sensors.

### Exemplification

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### EXAMPLE 1

The present example describes a dual-chamber element in accordance with some embodiments of the present disclosure.

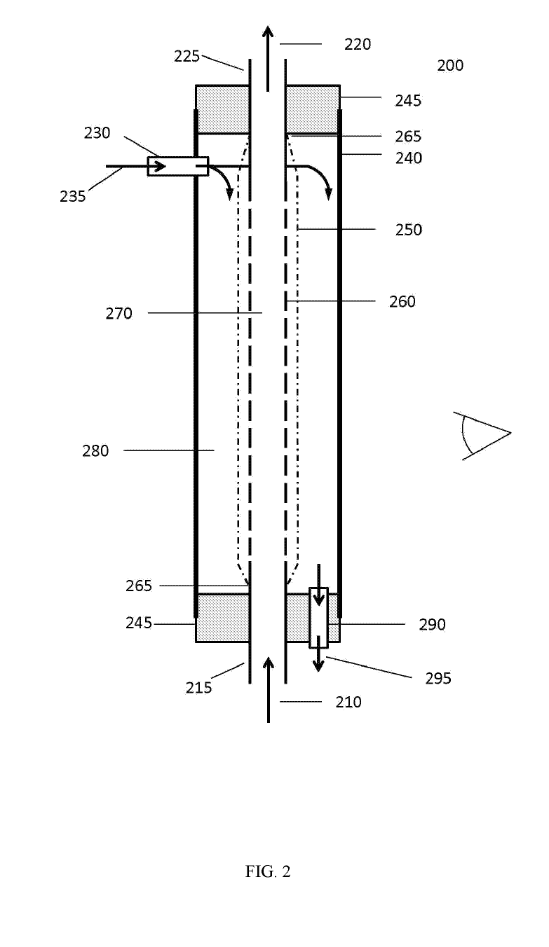

Referring to FIG. 2, a dual-chamber element 200 of an automated silk purification system in accordance with some embodiments is shown. The dual-chamber element 200 is substantially tubular. A dissolved silk fibroin solution 210 is introduced, enters, or flows into a first chamber 270 of the dual-chamber element 200 through an entrance 215. The dissolved silk fibroin solution 210 includes, for example, dissolved silk fibroin, salts (e.g. LiBr), solvents, contaminants, and/or ions. The first chamber 270 includes a permeable tube 260 that extends from the entrance 215 through to the exit 225. The permeable tube 260 is acrylic. A porous membrane 250 is a SnakeSkin dialysis membrane. The porous membrane 250 surrounds the permeable tube 260. The permeable tube 260 provides support and points of attachment 265 to which the porous membrane 250 is secured to the permeable tube 260. The dissolved silk fibroin solution 210 is introduced, enters, or flows into the first chamber 270. The dissolved silk fibroin solution 210 passes through the permeable tube 260 and fills the first chamber 270. The filled volume of the first chamber expands and is defined by the porous membrane 250.

The dissolved silk fibroin solution 210 flows through the first chamber 270 and out of the first chamber 270 at the exit 225. While flowing through the first chamber 270, the dissolved silk fibroin solution 210 contacts the porous membrane 250 and the dissolved silk fibroin solution 210 exerts a force normal to the porous membrane 250. The porous membrane 250 allows crossflow filtration of components of the silk fibroin solution, such as LiBr, water and other salts, solvents, contaminants, and/or ions while retaining the dissolved silk fibroin. The second chamber 280 of the dual-chamber element 200 is defined by an outer wall 240. The outer wall 240 is acrylic. The second chamber 280 of the dual-chamber element 200 is also defined by a pair of ends portions 245 that seal the second an outer wall 240. The permeable tube 260 passes concentrically through a surrounding outer wall 240. The permeable tube 260 is anchored by waterproof elastomeric end portions 245. The porous membrane 250 is slightly larger than the permeable tube 260. The end portions 245 also seal around a solid portion of each of the entrance 215 and exit 225 of the permeable tube 260. The second chamber 280 also includes an entrance port 230 for input of a dialysate 235. The dialysate 235 is milli-Q water. The milli-Q water 235 is gravity-fed. A manual valve on the drain line (not shown) is adjusted to control a flow rate. The counter-flow of water against a dissolved silk fibroin solution 210 containing LiBr causes effective removal of the LiBr and exchange with distilled water. The second chamber 280 also includes an exit port 290 for output of a permeate 295. The permeate 295 is an aqueous solution including salts (e.g. LiBr), solvents, contaminants, and/or ions. The flow 295 exiting the second chamber 280 at the exit port is emptied to a laboratory drain. A peristaltic pump (not shown) controls the flow rate of the dissolved silk fibroin solution 210.

A purified dissolved silk fibroin solution 220 exits the first chamber 270 at the exit 225.

The dual-chamber element 200 also includes monitoring, as shown, for example cameras, sensors, etc.

### EXAMPLE 2

The present example describes of a dual-chamber element in accordance with some embodiments of the present disclosure.

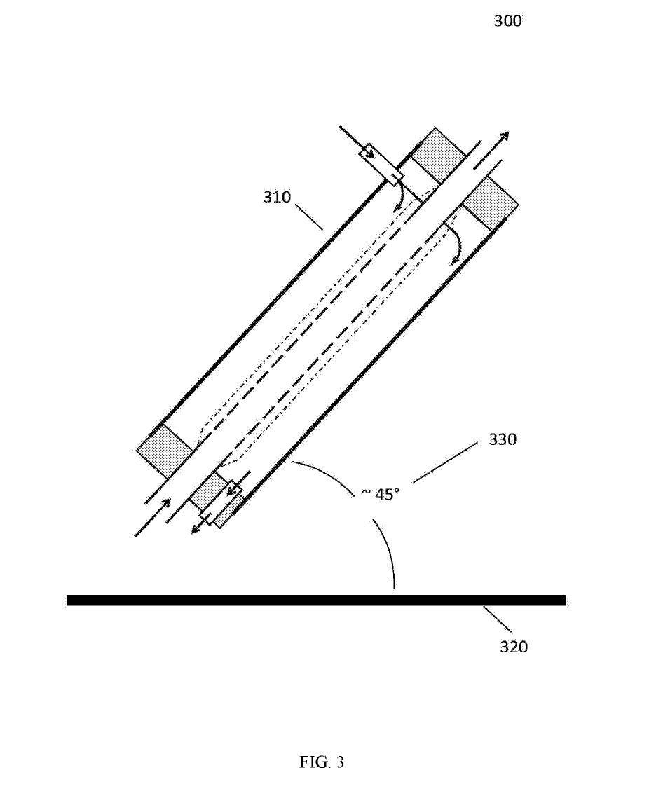

Referring to FIG. 3, a dual-chamber element 300 of an automated silk purification system in accordance with some embodiments is shown. The dual-chamber element 300 is substantially tubular. An outer wall 310 of the dual-element chamber 300 is shown tilted at an angle 330 relative to a surface 320. The angle 330 is shown as about 45°.

### EXAMPLE 3

The present example describes two silk dual-chamber elements connected in series in accordance with some embodiments of the present disclosure.

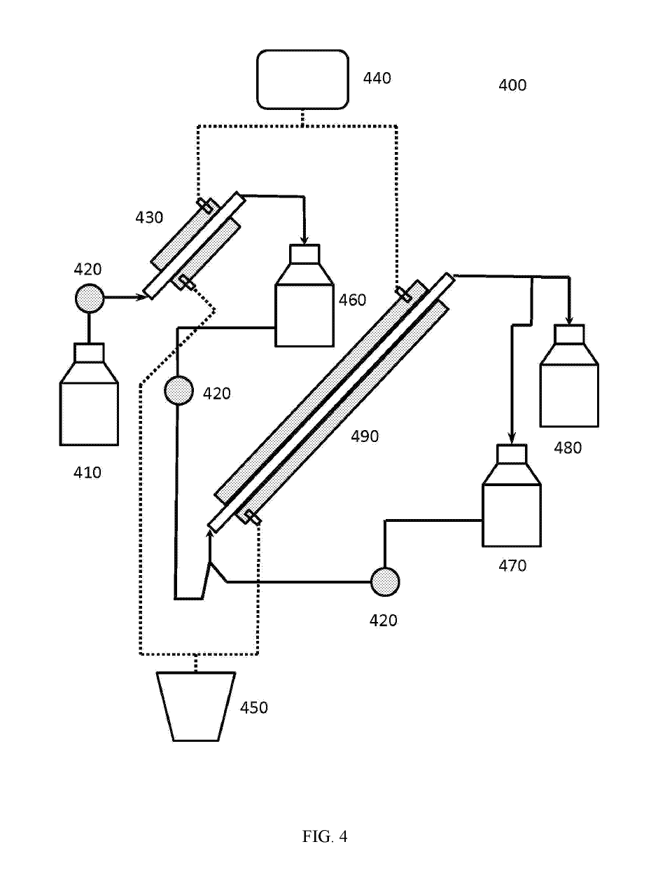

Referring to FIG. 4, an automated silk purification system 400 in accordance with some embodiments is shown. The silk solution purification system 400 shows two dual-chamber elements, a first dual-chamber element 430 and a second dual-chamber element 490. The first dual-chamber element 430 is about 30 cm long. The second dual-chamber element 490 is about 1 m long. The two dual-chamber elements 430 and 490 are both tilted. A first silk fibroin solution reservoir 410 contains a dissolved silk fibroin solution. The reservoir 410 is connected to a peristaltic pump 420. The pump 420 pumps the silk fibroin solution from the reservoir 410 through a first chamber of the first dual-chamber element 430. A milli-Q water supply 440 is gravity-fed to a second chamber of the first dual-chamber element 430. An output of a first purified silk fibroin solution enters a second silk fibroin solution reservoir 460.

The second silk fibroin solution reservoir 460 is connected to a peristaltic pump 420. The pump 420 pumps the silk fibroin solution from the reservoir 460 through a first chamber of the second dual-chamber element 490. A milli-Q water supply 440 is gravity-fed to a second chamber of the second dual-chamber element 490. An output of a the second purified silk fibroin solution enters a third silk fibroin solution reservoir 470 and/or fourth silk fibroin solution reservoir 480. The fourth silk fibroin solution reservoir 480 stores a purified silk fibroin solution.

The flow exiting the second chamber of the first dual-chamber element 430 and the second dual-chamber element 490 is emptied to a laboratory drain 450.

The third silk fibroin solution reservoir 470 is connected to a peristaltic pump 420. The pump 420 pumps the silk fibroin solution from the reservoir 470 through a first chamber of the second dual-chamber element 490. A milli-Q water supply 440 is gravity-fed to a second chamber of the second dual-chamber element 490 and is emptied to a laboratory drain 450. An output of a the second purified silk fibroin solution enters a third 470 and/or fourth 480 silk fibroin solution reservoir. The fourth silk fibroin solution reservoir 480 stores a purified silk fibroin solution.

An addition of the second tube allows for a more complete dialysis cycle for silk fibroin solution that passes through the entire length of both tubes.

### EXAMPLE 4

The present example describes silk fibroin solutions purified in a element system in accordance with some embodiments of the present disclosure and as shown in Example 3.

Neutron Activation Analysis was utilized to detect Bromine and Inductively Coupled Plasma Mass Spectrometry was utilized to detect Lithium in samples taken throughout the prototype process. Neutron Activation Analysis (NAA) is a sensitive multi-element analytical technique that was used to pick up all forms of Br in silk samples. Being a more sensitive technique than Ion Chromatography, NAA can detect elements below 5 ppm. Inductively Coupled Plasma Mass Spectrometry (ICP-MS) is highly sensitive and capable of detecting many metals and several non-metals to low concentrations. Results from the Li and Br testing are shown in Table 1.

TABLE 1
Lithium Bromide analysis

| | Mass | Bromine | Lithium |
|---|---|---|---|
| Sample | (g) | [wt %] (ppm) | (ppm) |
| Control | 1.169 | 9.1 (+/−0.319) | 71 |
| (via dialysis cassette) | | | |
| Silk Fibroin Solution Reservoir | 1.557 | >100000 | 26500 |
| (Ex. 3 FIG. 4-410) | | | |
| Initial Dissolved silk | | | |
| fibroin solution | | | |
| Silk Fibroin Solution Reservoir | 1.195 | 12500 (+/−375) | 2160 |
| (Ex. 3 FIG. 4-460) | | | |
| Silk Fibroin Solution that is | | | |
| Collected after passing through | | | |
| the Short Dual-Tube Element | | | |
| Silk Fibroin Solution Reservoir | 1.115 | 2.6 (+/−0.130) | 66 |
| (Ex. 3 FIG. 4-470) | | | |
| Silk Fibroin Solution Collected | | | |
| after passing through the Long | | | |
| Dual-Tube Element | | | |
| Silk Fibroin Solution Reservoir | 1.198 | 0.8 (+/−0.080) | 39 |
| (Ex. 3 FIG. 4-480) | | | |
| Silk Fibroin Solution Collected | | | |
| after twice passing through the | | | |
| Long Dual-Tube Element | | | |

The “Control” sample is a control Silk Fibroin Solution sample that was prepared using the standard protocol (i.e. processing using a dialysis cassette). In the standard process after dialysis, approximately 9.1 ppm and 71 ppm of Bromine and Lithium remain, respectively. Given that the process was performed according to the standard protocol, these results are assumed to be in an acceptable range for most applications of Silk Fibroin Solution.

An as-dissolved silk fibroin solution is shown to exceed the limits of the NAA detection technique, which is above 100000 ppm. The Lithium content was detected at 26500 ppm.

After undergoing dialysis through the short dual-element chamber 430, the Bromine and Lithium content present in reservoir 460 dropped to 12500 ppm and 2160 ppm, respectively. After then progressing through the long dual-element chamber 490, the levels in reservoir 470 dropped further to 2.6 ppm (Bromine) and 66 ppm (Lithium). It is interesting to note that these levels are below the levels that remain after the standard protocol is followed. By passing the solution back through the long dual-element chamber 490, the levels in reservoir 480 decreased to their lowest levels of 0.8 ppm (Bromine) and 39 ppm (Lithium). The SnakeSkin dialysis membrane of the silk solution purification system is effective at dialyzing the silk fibroin solution and removing the Lithium and Bromine that were added during the dissolving stage. It is shown that only a single pass through the short and long TFF tubes is necessary and that the dialysis process can be done in a continuous fashion (with the understanding that a fixed starting volume of dissolved silk is provided and a fixed volume of dialyzed silk fibroin solution is produced). The entire process took less than 48 hours and resulted in about a liter of a purified silk fibroin solution with a concentration of about 4.5% w/v.

### EXAMPLE 5

The present example describes of a dual-chamber element in accordance with some embodiments of the present disclosure.

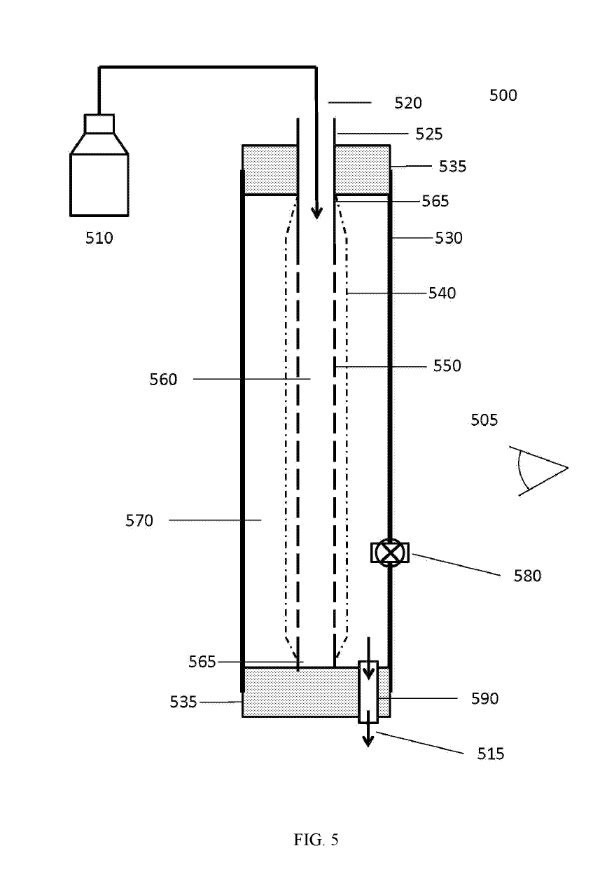

Referring to FIG. 5, a silk solution concentrating system 500 in accordance with some embodiments is shown. The silk solution concentrating system 500 includes a dual-chamber element 505. The dual-chamber element 505 is substantially tubular. A silk fibroin solution reservoir 510 stores a silk fibroin solution 520. The silk fibroin solution 520 previously was purified. The purified silk fibroin solution 520 includes, for example, dissolved silk fibroin. The first chamber 560 includes a permeable tube 550 that extends from the entrance 525. The permeable tube 550 is acrylic. A porous membrane 540 is a SnakeSkin dialysis membrane. The porous membrane 540 surrounds the permeable tube 550. The permeable tube 550 provides support and points of attachment 565 to which the porous membrane 540 is secured to the permeable tube 550. The purified silk fibroin solution 520 is gravity fed into the first chamber 560 of the dual-chamber element 505 through an entrance 525. The purified silk fibroin solution 520 passes through the permeable tube 540 and fills the first chamber 560. The filled volume of the first chamber expands and is defined by the porous membrane 540.

The second chamber 570 of the dual-chamber element 505 is defined by an outer wall 530. The outer wall 530 is acrylic. The second chamber 570 of the dual-chamber element 505 is defined by a pair of ends portions 535 that seal the second an outer wall 530. The permeable tube 550 passes concentrically through a surrounding outer wall 530. The permeable tube 550 is anchored by waterproof elastomeric end portions 535. The porous membrane 540 is slightly larger than the permeable tube 550. The end portions 535 seals around a solid portion of opposite an entrance 525 of the permeable tube 550.

The purified silk fibroin solution 520 is gravity fed into the first chamber 560. The purified silk fibroin solution 520 contacts the porous membrane 540. The purified silk fibroin solution 520 exerts a force normal to the porous membrane 540. The porous membrane 540 allows crossflow filtration of components of the silk fibroin solution, such solvents, for example water while retaining a dissolved concentrated silk fibroin. A concentrated dissolved silk fibroin solution is retained in the second chamber 570.

A manual valve 580 is shown attached to an out wall 530. The valve 580 is for removing a concentrated silk fibroin solution from the concentrating system. The second chamber 570 includes a gas, such as air.

The second chamber 570 also includes an exit port 590 for solvent output. The flow 515 exiting the second chamber 570 at the exit port is emptied to a laboratory drain.

While the purified silk fibroin solution 520 is in the second chamber, water is removed from the solution. The rate is dependent on the humidity, temperature, and air flow in the surrounding environment. As water is removed and the column height becomes lower, fresh solution is automatically supplied as the top of the dual-chamber element. The solution at the bottom of the second chamber 570 has a higher concentration, the concentrated solution is near the bottom because it was in the column the longest and the silk fibroin is denser than water.

The concentrated purified silk fibroin solution taken from the silk solution concentrating system 500 has been concentrated from the 4.5% w/v level to over 10% w/v.

The dual-chamber element 505 also includes monitoring, as shown, for example cameras, sensors, etc.

### OTHER EMBODIMENTS AND EQUIVALENTS

While the present disclosures have been described in conjunction with various embodiments, and examples, it is not intended that they be limited to such embodiments, or examples. On the contrary, the disclosures encompass various alternatives, modifications, and equivalents, as will be appreciated by those of skill in the art. Accordingly, the descriptions, methods and diagrams of should not be read as limited to the described order of elements unless stated to that effect.

Although this disclosure has described and illustrated certain embodiments, it is to be understood that the disclosure is not restricted to those particular embodiments. Rather, the disclosure includes all embodiments, that are functional and/or equivalents of the specific embodiments, and features that have been described and illustrated. Accordingly, for example, methods and diagrams of should not be read as limited to a particular described order or arrangement of steps or elements unless explicitly stated or clearly required from context (e.g., otherwise inoperable). Moreover, the features of the particular examples and embodiments, may be used in any combination. The present disclosure therefore includes variations from the various examples and embodiments, described herein, as will be apparent to one of skill in the art.

What is claimed is:

1. An automated system, comprising:
at least one dual-chamber element, comprising first and second chambers separated from one another by a porous membrane,
wherein the at least one dual-chamber element is dimensioned and the system is arranged and constructed so that when a dissolved silk fibroin solution travels into or through the first chamber, salts, contaminants, solvents, and/or ions from the dissolved silk fibroin solution cross the porous membrane into a dialysate in the second chamber, and silk proteins from the dissolved silk fibroin solution are retained in a retentate in the first chamber, thereby filtering the dissolved silk fibroin solution, and
wherein a pressure and/or a flow of the dissolved silk fibroin solution as it travels is below a threshold that induces silk protein aggregation.

2. The automated system of claim 1, wherein each of the first and second chambers are substantially tubular.

3. The automated system of claim 1 or claim 2, wherein the second chamber substantially surrounds the first chamber so that the second and first chambers are outer and inner chambers, respectively.

4. The automated system of any of the preceding claims, wherein the porous membrane has a tubular shape that defines the inner chamber.

5. The automated system of any of the preceding claims, comprising at least two dual-chamber elements.

6. The automated system of claim 5, wherein the at least two dual-chamber elements are arranged in series.

7. The automated system of claim 5 or claim 6, further comprising an mixing stage between the two dual-chamber elements.

8. The automated system of any of claims 5-7, wherein each of the at least two dual-chamber elements is a different length.

9. The automated system of any of claims 5-8, wherein a length of a first chamber of the at least two dual-chamber elements is about 1 m, about 95 cm, 90 cm, about 85 cm, about 80 cm, about 75 cm, about 70 cm, about 65 cm, about 60 cm, about 55 cm, about 50 cm, about 45 cm, about 40 cm, about 35 cm, about 30 cm, about 25 cm, about 20 cm, about 15 cm, about 10 cm, and about 5 cm.

10. The automated system of any of claims 5-8, wherein a length of a second chamber of the at least two dual-chamber elements is about 50 cm, about 45 cm, about 40 cm, about 35 cm, about 30 cm, about 25 cm, about 20 cm, about 15 cm, about 10 cm, about 5 cm, about 4 cm, about 3 cm, about 2 cm, and about 1 cm.

11. The automated system of any of the preceding claims, wherein the porous membrane has pores sized to retain proteins between about 1 kDa and about 100 kDa.

12. The automated system of any of the preceding claims, wherein the dissolved silk fibroin solution has a viscosity between about 1.5 cP and 20 cP.

13. The automated system of any of the preceding claims, wherein the dissolved silk fibroin solution has a flow rate in a range of about 0.01 ml per minute to about 0.5 ml per minute for a volume within a range of about 1 ml to about 100 liters.

14. The automated system of any of the preceding claims, wherein the porous membrane comprises one or more members selected from a group consisting of a semi-permeable membrane, a selectively permeable membrane, a dialysis membrane, cellulose tubing, regenerated cellulose tubing, or SnakeSkin tubing.

15. The automated system of any of the preceding claims, further comprising a dialysate solution in the second chamber.

16. The automated system of claim 15, wherein the dialysate solution is a counter-flow fluid that flows in the second chamber in a direction opposite to that of the dissolved silk fibroin solution.

17. The automated system of claim 16, wherein the counter-flow fluid is one or more members selected from the group consisting of water, polyethylene oxide, glycerol, polyvinyl alcohol, or hygroscopic polymer fluids.

18. The automated of claim 15 or claim 16, wherein the salts, contaminants, solvents, and/or ions from the dissolved silk fibroin solution are removed when the counter-flow fluid is extracted from the second chamber.

19. The automated system of any of the preceding claims, wherein each of the at least one dual-chamber elements is tilted at an angle relative to a vertical reference axis.

20. The automated system of claim 19, wherein the angle relative to the vertical axis is from about 15° to about 85°.

21. The automated system of claim 19 or claim 20, wherein the angle relative to the vertical axis is about 45°.

22. The automated system of any of the preceding claims, wherein the salt comprises one or more members selected from the group consisting of lithium thiocyanate (LiSCN), sodium thiocyanate (NaSCN), calcium thiocynanate (Ca(SCN)₂), magnesium thiocyanate (MG(SCN)₂), calcium chloride (CaCl₂), lithium bromide (LiBr), zinc chloride (ZnCl₂), magnesium chloride (MgCl₂), copper nitrate (Cu(NO₃)₂), copper ethylene diamine (Cu(NH₂CH₂CH₂NH₂)₂(OH)₂), and Cu(NH₃)₄(OH)₂.

23. The automated system of any of the preceding claims, further comprising a pump to remove air pockets that form at a top of the at least two dual-chamber elements.

24. The automated system of any of the preceding claims, wherein the dissolved silk fibroin solution has a kinematic viscosity between about 2 centistokes and about 20 centistokes.

25. A post-dialysis silk concentrating system, comprising:
a dual-chamber element, comprising first and second chambers separated from one another by a porous membrane,
so that when a purified silk fibroin solution is gravity fed into the first chamber, the purified silk fibroin solution separates and a concentrated purified silk fibroin solution is contained in the first chamber and a solvent cross the porous membrane into the second chamber.

26. The post-dialysis silk concentrating system of claim 25, wherein the second chamber substantially surrounds the first chamber so that the second and first chambers are outer and inner chambers, respectively.

27. The post-dialysis silk concentrating system of claim 26, further comprising a gas in the outer chamber.

28. The post-dialysis silk concentrating system of claim 27, wherein the gas is air.

29. The post-dialysis silk concentrating system of any of claims 26-28, wherein the concentrated purified silk fibroin solution separates forming a gradient within the inner chamber, wherein the gradient is characterized by a relatively higher concentrated purified silk fibroin solution on a bottom of the at least one dual-chamber element and a relatively lower concentrated purified silk fibroin solution on a top of the at least one dual-chamber element.

30. The post-dialysis silk concentrating system of claim 29, wherein the relatively higher concentrated purified silk fibroin solution is between about 30% w/v to about 50% w/v.

31. The post-dialysis silk concentrating system of any of claims 26-30, further comprising at least one sensor to in situ monitor a concentration of the concentrated purified silk fibroin solution throughout the gradient.

32. The post-dialysis silk concentrating system of any of claims 26-31, further comprising a camera for process monitoring.

33. The post-dialysis silk concentrating system of any of claims 26-32, further comprising at least one outlet port in the at least one dual-chamber element.

34. A system, comprising:
at least two dual-chamber elements, comprising first and second chambers separated from one another by a porous membrane,
wherein the at least one dual-chamber element is dimensioned and the system is arranged and constructed so that when a dissolved silk fibroin solution flows into or through the first chamber of a first dual-chamber element, salts, contaminants, solvents, and/or ions from the dissolved silk fibroin solution cross the porous membrane into the second chamber, and silk proteins from the dissolved silk fibroin solution are retained in a retentate solution in the first chamber, thereby filtering the dissolved silk fibroin solution,
wherein a pressure and/or a flow of the dissolved silk fibroin solution as it travels is below a threshold that induces silk protein aggregation, and
wherein when the purified silk fibroin solution is gravity fed into another dual-chamber element, the silk fibroin solution separates forming a concentrated purified silk fibroin solution.

35. The system of claim 34, wherein the dissolved silk fibroin solution has a viscosity between about 1.5 cP and 20 cP.

36. The system of claim 34 or claim 35, wherein the dissolved silk fibroin solution has a flow rate in a range of about 0.1 0.01 ml per minute to about 0.5 ml per minute for a volume within a range of about 1 ml to about 100 liters,

37. The system of any of claims 34-36, wherein the concentrated purified silk fibroin solution separates forming a gradient within the inner chamber, wherein the gradient is characterized by a relatively higher concentrated purified silk fibroin solution on a bottom of the at least one dual-chamber element and a relatively lower concentrated purified silk fibroin solution on a top of the at least one dual-chamber element.

38. The system of claim 37, wherein the relatively higher concentrated purified silk fibroin solution is between about 30% w/v to about 50% w/v.

39. The system of any of claims 34-38, further comprising at least one sensor to in situ monitor a concentration of the concentrated purified silk fibroin solution throughout the gradient.

40. The system of any of claims 34-39, further comprising at least one outlet port in the at least one dual-chamber element.

41. The system of any of claims 34-40, further comprising a camera for process monitoring.

42. A method for purifying a dissolved silk fibroin solution with an automated system, the method comprising:
providing a dissolved silk fibroin solution;
flowing the dissolved silk fibroin solution through the inner volume in a first direction; and
providing a counter-flow of a fluid in the outer volume in a direction opposing the flow of the dissolved silk fibroin solution,
so that when the dissolved silk fibroin solution flows through the inner volume and across the membrane, the membrane retains silk proteins within a retentate solution and the membrane allows salts and contaminants from the dissolved silk fibroin solution to cross into the outer volume in an exchange with a counter-flow fluid, and
wherein a pressure and/or a flow of the dissolved silk fibroin solution as it travels is below a threshold that induces silk protein aggregation
extracting the counter-flow fluid.

43. The method of claim 42, wherein the dissolved silk fibroin solution has a kinematic viscosity between about 2 centistokes and about 20 centistokes.

44. The method of claim 42 or claim 43, wherein the porous membrane is sized to retain proteins larger than 3500 Da.

45. The method of any of claims 42-44, wherein the extracting step further comprises steps of:
gravity feeding the retentate solution into a post-dialysis silk concentrating system, the system comprising:
a dual-chamber element, comprising first and second chambers separated from one another by a porous membrane,
so that when a purified silk fibroin solution is gravity fed into the first chamber, the purified silk fibroin solution separates from its solvent forming a concentrated purified silk fibroin solution.
